(19) United States
(12) Patent Application Publication
(10) Pub. No.: US 2012/0225019 A1
(43) Pub. Date: Sep. 6, 2012

(54) Dual Fluorescence Assay For Determining Viability Of Parasitic Or Non-Parasitic Worms

(76) Inventors: Emily Peak, Penpareau (GB); Karl Francis Hoffmann, Talybont (GB)

(21) Appl. No.: 13/496,733

(22) PCT Filed: Sep. 13, 2010
(86) PCT No.: PCT/GB2010/051527

§ 371 Date: May 23, 2012

(30) Foreign Application Priority Data
Sep. 16, 2009 (GB) 0916240.5
Jun. 4, 2010 (GB) 1009431.6

(51) Int. Cl. A61K 49/00; B01L 3/00

(52) U.S. Cl. 424/9.2; 424/9.6; 422/553

(57) ABSTRACT

A two colour fluorescent assay and a kit are described that permit the determination of live and dead parasitic or non-parasitic worms, for example helminths. The assay permits screening for the effect of one or more agents or an event on the viability of a parasitic or non-parasitic worm. In one example, the assay comprises (a) providing a sample comprising one or more parasitic or non-parasitic worms; (b) contacting the parasitic or non-parasitic worms with a concentration of fluorescein diacetate sufficient to yield detectable green fluorescence in any live parasitic or non-parasitic worms present in the sample and a concentration of propidium iodide sufficient to yield detectable red fluorescence in any dead parasitic or non-parasitic worms present in the sample; and (c) detecting the red and green fluorescence of the parasitic or non-parasitic worms. The assay further comprises contacting the parasitic or non-parasitic worms with one or more test agents, or subjecting the parasitic or non-parasitic worms to an event. The assay allows for a rapid and objective score of parasitic or non-parasitic worm death and survival and enables the performance of high- throughput screens for the identification of potential agents against diseases such as schistosomiasis. Also described are methods and kits for discriminatory analysis of parasitic or non-parasitic worm phenotype and/or metabolics using FTIR analysis.

### FIELD OF THE INVENTION

This invention describes the use of two fluorescent dyes in an assay that permits the determination of live and dead parasitic and non-parasitic worms, for example helminths and the use of the assay in the screening of agents against parasitic and non-parasitic worm, for example helminth viability. The assay permits high- throughput screening for the viability of parasitic and non-parasitic worms, in particular helminths of the phyla Platyhelminthes and Nematoda. This invention also describes methods for discriminatory analysis of parasitic and non-parasitic worm phenotype and/or metabolic changes.

### BACKGROUND OF THE INVENTION

Schistosomiasis is a vascular parasitic disease in humans caused by infection with blood flukes of the Schistosoma species. Infection with the parasitic trematode Schistosoma mansoni causes a wide range of quantifiable clinical pathologies (C. H. King, K. Dickman, and D. J. Tisch, Lancet 365 (9470), 1561 (2005), which collectively leads to the death of approximately 200,000 individuals per annum (A. Fenwick, L. Savioli, D. Engels et al., Trends Parasitol 19 (11), 509 (2003)). The treatment of schistosomiasis in humans relies on the use of one drug (Praziquantel). Resistance to this drug has the potential to severely hamper our ability to cure this disease and so lends an urgency to the search for novel drug targets.

The recent availability of the draft genomes of both Schistosoma mansoni and S. japonicum, as well as multiple reports describing the utilization of numerous functional genomic tools (e.g., C. H. Hokke, J. M. Fitzpatrick, and K. F. Hoffmann, Trends Parasitol 23 (4), 165 (2007);P. 3. Brindley and E. J. Pearce, Int J Parasitol 37 (5), 465 (2007)), has now provided the technological framework for a renaissance in drug target and vaccine discovery research (D. G. Colley and W. E. Secor, PLoS Negl Trop Dis 1 (2), e32 (2007)). A major bottleneck in converting schistosome phenotypic discovery into applied therapeutic products, however, is the lack of appropriate methods for quantifying, in a high-throughput manner, individual gene function or small compound effect on parasite survival. Current methods for detecting schistosome viability rely on qualitative microscopic criteria (K. F. Hoffmann, S. L. James, A. W. Cheever et al., J Immunol 163 (2), 927 (1999);A. N. Kuntz, E. Davioud-Charvet, A. A. Sayed et al., PLoS Med 4 (6), e206 (2007); D. Gold, Parasitol Res 83 (2), 163 (1997)), which require an understanding of parasite morphology, and most importantly, can be subjectively interpreted. These methods are therefore unsuitable for high-throughput identification of novel anti-schistosome drugs.

It is common to refer to cells that have an intact cell membrane as “viable” cells and cells where the membrane has been irreversibly disrupted by a cytotoxic reagent as “dead” or “non-viable” cells. It is known that fluorescent dyes can be used for the detection of viable and dead cells. Propidium iodide (PI) is a fluorescent dye that is membrane impermeant and generally excluded from viable cells. Dead cells or cells whose membrane integrity has been damaged can be penetrated by PI where the PI binds to nucleic acids resulting in a red fluorescence. Fluorescein diacetate (FDA) is membrane permeable and virtually non fluorescent. FDA can pass through cell membranes and is hydrolyzed by intraceliular esterases of a living cell to produce the green fluorescent compound fluorescein, which is trapped within the living cell. Thus, PI is an effective stain to identify non-viable cells and FDA is an effective stain to identify viable cells.

FDA and PI have been used together in a method to determine cell viability (Jones, Kenneth H. and Senft, James A., J. Histochem and Cytochem., 33, 1, pp 77-70, (1985)). In the method described, cells from mice spleen were stained with FDA and PI and applied to a microscope slide for analysis.

Abdulla, Maha-Hamadien, et al., “Drug Discovery for Schistosomiasis: Hit and Lead Compounds Identified in a Library of Known Drugs by Medium-Throughput Phenotypic Screening.” PLoS, 3, (7), e478 (2009), describe a medium-throughput phenotypic screen for identifying potential compounds against Schistosomiasis. The screen comprises three-stages. In the first stage, compounds are screened against the schistosomula stage of the parasite and hits from the first stage are screened against adult parasites in the second stage. Finally, hits from the second stage are screened against a murine model of the disease in the third stage. The screen described employs a visual classification of phenotypes and is therefore not suitable for high-throughput screening of compounds and prone to subjectivity. It is to be noted that the authors state that their attempts to develop a screening assay using nuclear dyes have been unsuccessful.

There is, therefore, a need for improved methods of screening for parasitic and non-parasitic worm, for example helminth viability and for screening for agents against parasitic and non-parasitic worm, for example helminth viability.

In one aspect, the invention describes a reproducible fluorescent-based bioassay to improve detection of parasitic and non-parasitic worm, for example helminth viability, in particular the viability of organisms within the phyla Platyhelminthes and Nematoda, which is high-throughput, quantitative and provides non-subjective readouts of helminth survival during in vitro culture. The versatility of the assay in detecting schistosome survival in response to drug- targeting of thioredoxin glutathione reductase (TGR) function is demonstrated. Furthermore, this assay is utilised to confirm/test the anti-schistosomula activity of gambogic acid, salinomycin, ethinyl estradiol, fluoxetine hydrochloride, bepridil, ciclopirox and miconazole nitrate. By use of the method according to the invention, inter-laboratory comparisons of in vitro parasite manipulations can be routinely and rapidly performed, vastly accelerating the search for novel anti-schistosomal lead targets.

Definitions

The use of the term “parasitic and non-parasitic worms” includes organisms which are found within the Phyla Platyhelminthes and Nematoda. Examples of parasitic worms include members of the following genera (not inclusive): Schistosoma, Fasciola, Opisthorchis, Paragonimus, Clonorchis, Haemonchus (e.g. Haemonchus contortus), Ostertagia, Necator, Trichuris, Taenia, Dicrocoelium, Echinococcus, Hymenolepis, Ancylostoma, Ascaris, Strongyloides, Wucheria, Toxocara, Onchocerca, Brugia, Trichostrongylus, Gyrodactylus and Dirofilaria. Examples of non-parasitic worms include members of the following genera (not inclusive): Caenorhabditis, Dugesia, Planaria and Schmidtea.

The use of the term “helminths” is to be construed to cover organisms within the two phyla Platyhelminthes and Nematoda. All stages of the platyhelminth and nematode life cycle, including the egg, larval and other immature adult stages and mature egg-laying adult stage are included in the term. For example, for Platyhelminthes within the genus Schistosoma, the larvae, known as cercariae, and schistosomula are included. In other examples, for Haemonchus contortus, the larvae, known as Haemonchus contortus L3 larvae, are included. In some examples, the methods of the present invention can be applied to adult male and female helminths.

The term “test agent” refers to an agent that is to be screened in one or more of the assays described herein. The agent can be any chemical compound, such as an organic compound or nucleic acid (i.e. dsRNA or siRNA), and can exist as a single isolated compound or can be a member of a combinatorial library.

The term “event” refers to exposing the parasitic or non-parasitic worm, for example helminth to a change in its environment. The event may be adding a biological agent such as any cell, tissue, organism, or extracts thereof, metabolites from cells or tissues, immunoglobulins or other molecules, a temperature change or a barometric change.

The use of the terms “a” and “an” and “the” and similar referents in the context of describing the invention (especially in the context of the following claims) are to be construed to cover both the singular and the plural, unless otherwise indicated herein or clearly contradicted by context.

Within this specification, the term “about” means plus or minus 20%, more preferably plus or minus 10%, even more preferably plus or minus 5%, most preferably plus or minus 2%.

Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed.

Within this specification, the terms “comprises” and “comprising” are interpreted to mean “includes, among other things”. These terms are not intended to be construed as “consists of only”.

### DESCRIPTION OF THE DRAWINGS

FIG. 1 demonstrates fluorescein diacetate (FDA) and propidium iodide (PI) can be used to differentially quantify schistosomula viability. Mechanically-transformed schistosomula were prepared, heat-killed (dead) or left untreated (live) and stained with FDA, PI or both fluorophores according to the Methods. Epi-fluorescent and plane polarized microscopy was used to visualize uptake of fluorophores and examine schistosomula morphology. (A) Dead schistosomula stained with PI and detected by a rhodamine filter (536 nm), (B) Dead schistosomula visualized by polarized light, (C) Live schistosomula stained with FDA and detected by a FITC filter (494 nm), (D) Live schistosomula visualized by polarized light (E) Mixtures of dead and live schistosomula simultaneously stained with PI/FDA and detected by a rhodamine filter, (F) Mixtures of dead and live schistosomula simultaneously stained with PI/FDA and detected by a FITC filter, (G) Differential detection of PI-positive dead and FDA-positive live schistosomula by superimposition of both 536 nm and 494 nm epifluorescent spectra, (H) Differential morphology of dead and live schistosomula detected by polarized light. PI was found to stain only dead schistosomula, while FDA exhibited preferential staining for live schistosomula. Differential staining was supported by observations of schistosomula shape and motility. Heat-killed PI-positive schistosomula were uniform in shape and size and displayed no movement, whereas physiologically normal FDA-positive schistosomula displayed a variety of shapes and sizes as a result of movement during in vitro culturing.

FIG. 2 demonstrates that a kinetic study of FDA and PI emission reveals the optimal timeframes to collect fluorescent signals from stained schistosomula samples. Mechanically-transformed schistosomula were cultured for 24 hr, heat killed (dead) or left untreated (live), simultaneously stained with PI and FDA and subjected to fluorescent readings at minute intervals. PI fluorescence (544 nm) was measured over 120 min whereas FDA fluorescence (485 nm) was measured over 51 min. (A) PI fluorescence data collected from a 96-well microtiter plate, (B) PI data collected from a 384-well microtiter plate, (C) FDA data collected from a 96-well microtiter plate and (D) FDA data collected from a 384-well microtiter plate. All fluorescent readings were obtained from a BMG Labtech Polarstar Omega microtiter plate reader. As indicated, each line represents either fluorescent data originating from wells containing dead schistosomula, fluorescent data originating from wells containing live schistosomula, fluorescent data from wells containing an equal number (mixed) of dead and live schistosomula or fluorescent data originating from wells containing no schistosomula (media). * Indicates chosen time point for collecting PI data and ♦ indicates chosen time point for collecting FDA data in subsequent experiments. All experiments were performed at least three times. Schistosomula were plated at 1000 parasites/well in the 96-well microtiter plate format and 200 parasites/well in the 384-well microtiter plate format. From statistical analyses and fluorescent ratio comparisons, the optimal time to measure PI emission is anytime after 4 minutes, while the optimal time to measure FDA emission is between 3 and 12 minutes.

FIG. 3 demonstrates that a schistosomula titration series reveals the optimal number of parasites to be used for PI and FDA fluorescent detection in both 96-well and 384-well microtiter plates. Mechanically-transformed schistosomula were cultured for 24 hr, heat killed (dead) or left untreated (live) and stained with either PI (dead parasites) or FDA (live parasites). (A) Dead schistosomula were titrated from 5000 to 36 parasites per well (in triplicate) in a 96-well microtiter plate and PI fluorescence (544 nm) obtained at 20 min, (B) Dead schistosomula were titrated from 1000 to 8 parasites per well (in triplicate) in a 384-well microtiter plate and PI fluorescence (544 nm) obtained at 20 min, (C) Untreated, live schistosomula were titrated from 5000 to 36 parasites per well (in triplicate) in a 96-well microtiter plate and FDA fluorescence (485 nm) measured at 5 min, (D) Untreated, live schistosomula were titrated from 1000 to 8 parasites per well (in triplicate) in a 384-well microtiter plate and FDA fluorescence (485 nm) measured at 5 min. All fluorescent readings were collected from a BMG Labtech Polarstar Omega microtiter plate reader. These results are representative of two independent experiments.

FIG. 4 demonstrates that dual FDA and PI staining of schistosomula samples allows for fluorescent quantification of parasite viability. Mechanically-transformed schistosomula were cultured for 24 hr, heat killed (dead) or left untreated (live), distributed into wells at pre-defined percentages (100% live, 75% live, 50% live, 25% live 0% live) and simultaneously stained with PI and FDA. (A) Pre-defined percentages of live and dead schistosomula (1000 parasites per well, in triplicate) distributed into a 96-well microtiter plate with PI fluorescence (544 nm) measured at 20 min, (B) Pre-defined percentages of live and dead schistosomula (1000 parasites per well, in triplicate) distributed into a 96-well microtiter plate with FDA fluorescence (485 nm) measured at 5 min, (C) Pre-defined percentages of live and dead schistosomula (200 parasites per well, in triplicate) distributed into a 384-well microtiter plate with PI fluorescence (544 nm) detected at 20 min, (D) Pre-defined percentages of live and dead schistosomula (200 parasites per well, in triplicate) distributed into a 384-well microtiter plate with FDA fluorescence (485 nm) detected at 5 min. (E) Schistosomula viability calculated from fluorescent data displayed in A and B, (F) Schistosomula viability calculated from fluorescent data presented in C and D. These results are representative of experiments performed three times.

FIG. 5 demonstrates dual-fluorescence viability determination of auranofin treated schistosomula. Mechanically-transformed schistosomula were cultured for 24 hr, incubated with different concentrations of auranofin for an additional 24 hr, washed and subsequently co-stained with both PI and FDA. PI-(544 nm, collected at 20 min) and FDA-(485 nm, collected at 5 min) fluorescence intensity units were converted into viability measures according to the formula described in the Methods. (A) Dose-dependent anti-schistosomula effect of auranofin as indicated by % viability. Treatments showing statistically significant differences in viability when compared to untreated (live) schistosomula are indicated with * (p<0.05) or ** (p<0.001). (B) Auranofin dose-response curve by which an LD₅₀ was calculated by plotting the probit transformation of the % viability to the Log₁₀ transformation of auranofin concentration. Dotted line indicates the average LD₅₀ value calculated from three replicates. (C) Light microscope image of schistosomula treated with 10 μM auranofin for 24 hr. (D) Light microscope image of untreated schistosomula. (E) Epi-fluorescent and (F) plane polarized light micrograph of schistosomula treated with 10 μM auranofin for 24 hr, then incubated with both PI and FDA. (G) Epi-fluorescent and (H) plane polarized light micrograph of schistosomula treated with 1 μM auranofin for 24 hr, then incubated with PI and FDA. ‘Dead’ represents schistosomula killed by heat-treatment and ‘0 μM’ auranofin represents schistosomula incubated with 1 (v/v) DMSO (auranofin solvent). These results are representative of experiments performed three times. Auranonfin induces a clear, titratable anti-schistosomula effect after 24 hours of culture.

FIG. 6 demonstrates application of the dual-fluorescent viability assay for determining the anti-schistosomula effect of selected compounds with previously-described or unknown activities. Mechanically-transformed schistosomula were cultured for 24 hr, incubated with compounds (10 μM) for an additional 24 hr, washed and subsequently co-stained with both PI and FDA. PI-(544 nm, collected at 20 min) and FDA-(485 nm, collected at 5 min) fluorescence intensity units were converted into viability measures according to the formula described in the Methods. (A) Calculated schistosomula viability in response to each compound tested. Representative epi-fluorescent and plane polarized microscope images of schistosomula treated with (B and G) gambogic acid, (C and H) sodium salinomycin, (D and I) niclosamide, (E and 3) praziquantel and (F and K) ciclopirox are indicated. Compounds designated as having ‘previously published activities’ (solid histograms—death, vertical lines within histograms—overactive, hatched lines within histograms—rounded) were selected from Abdulla, Maha-Hamadien, et al., “Drug Discovery for Schistosomiasis: Hit and Lead Compounds Identified in a Library of Known Drugs by Medium-Throughput Phenotypic Screening.” PLoS, 3, (7), e478 (2009) while those compounds indicated as having ‘unknown activities’ were selected from Berriman M., et al, “The genome of the blood fluke Schistosoma mansoni”, Nature, 460 (7253): 352-358 (2009)). These results are representative of two independent experiments.

FIG. 7 demonstrates that the dual fluorescence viability assay can be applied to adult male and female schistosomes. In FIG. 7(a), single adult male worms were placed into wells of a 96-well microtiter plate (1 worm/well) and dual stained with PI and FDA. FIG. 7(b) depicts a similar experiment performed with single adult female worms (1 worm/well). In both cases, when measuring PI fluorescence, dead worms fluoresced brightest when compared to live worms or culture media. FIG. 7(c) illustrates a similar experiment performed with 3 adult male worms/well in a 96-well microtiter plate and FIG. 7(d) illustrates 3 adult female worms/well. Again, dead parasites (regardless of gender) fluoresce brightest (in the PI channel) when compared to live parasites or media. FIG. 7(e-f) replicates these experiments in 384-well microtiter plates where 1 adult worm/well (male—7e; female—7f) was assayed for PI uptake and fluorescence. Again, dead parasites fluoresced brightest in the PI channel. FIGS. 7(g-l) represent the FDA readings from 1 adult male/well in a 96-well microtiter plate format (FIG. 7g), 1 adult female/well in a 96-well microtiter plate format (FIG. 7h), 3 adult males/well in a 96-well microtiter plate format (FIG. 7i), 3 adult females/well in a 96-well microtiter plate format (FIG. 7j), 1 male/well in a 384-well microtiter plate format (FIGS. 7k) and 1 female/well in a 384-well microtiter plate format (FIG. 7l). In all cases, live parasites stained brightest, in the FDA channel, when compared to dead parasites or media.

FIG. 8 demonstrates the influence of media components on FDA hydrolysis and schistosomula viability. FIG. 8(a) shows that the major source of FDA hydrolysis (in schistosomula culturing media) originates in the fetal calf serum supplement (dotted line, uppermost on graph). Other essential components (Pen-strep and L-glutamine; long dashed line, third from uppermost on graph) do not contribute to substantial increases in spontaneous FDA hydrolysis (over media control; solid black line). FIG. 8(b) shows that schistosomula are perfectly viable in minimal essential medium (DMEM+pen/strep+L-glutamine) for up to 48 hours (solid histogram) and the addition of FCS does not improve viability.

FIG. 9 illustrates that the dual-fluorescence bioassay can be completed in as little as 24 hours. Schistosomula were cultivated for either 3 or 24 hr (solid histogram), then incubated with test compounds or left untreated for 24 hr. No differences in viability were detected when either 3 or 24 hr schistosomula were used. This finding clearly indicates that, by using 3 hr cultured schistosomula, each dual fluorescent assay can be performed in 24 hr, greatly accelerating the throughput.

FIG. 10 shows how fourier transform infrared spectroscopy (FTIR) and metabolomics can be used to discriminate schistosomula displaying diverse phenotypes. Schistosomula cultured for 3 hr in 384-well microtiter plates were treated with 3 different compounds that induced a shape change or 4 compounds that induced overactivity. Culture supernatants were analyzed by FTIR for discriminatory metabolic peaks in the infrared spectra. Principle component analysis was applied to the raw data and discriminated the schistosomula displaying diverse phenotypes.

FIG. 11 shows photo images of treated L3 Haemonchus contortus larvae prior to the set up of FTIR. (a) DMSO treated control, (b) aldicarp, (c) ivermectin, (d) levamisole.

FIGS. 12A to 12C show FTIR results for the examples relating to Haemonchus contortus FTIR. WAD=worms cultured with aldicarb, WD=worms cultured with DMSO, WI=worms cultured with ivermectin, WL=worms cultured with levamisole, MAD=media containing aldicarb, MD=media containing DMSO, MI=media containing ivermectin, ML=media containing levamisole, SAD=supernatant from worms cultured with aldicarb, SD=supernatant from worms cultured with DMSO, SI=supernatant from worms cultured with ivermectin, SL=supernatant from worms cultured with levamisole. The circles on the DFA graphs represent mean group centres with confidence intervals of 95% (inner circle) and 99% (outer circle).

FIG. 12A shows worms results—good separation for ivermectin, levamisole and aldicarb treatments from the DMSO control treatment.

FIG. 12B shows media results—no separation between the treatments with DMSO, ivermectin, levamisole and aldicarb.

FIG. 12C shows supernatant results—showing no clear separation between any of the treatments.

### SUMMARY OF THE INVENTION

In one aspect, this invention provides a high-throughput screening method for determining parasitic and non-parasitic worm, for example helminth, viability by using two different colour fluorescent dyes.

The methods according to this aspect of the invention use two fluorogenic reagents, for example, PI and FDA, in combination. Following the use of the invention, the organisms of a given sample are stained either with one colour or another different colour. The two different dyes used emit fluorescent light at different wavelength ranges. In this manner, all organisms are detected and thus an absolute count of organisms and percent viability can be obtained.

The fluorescence can be measured using any means known in the art, for example by a plate reader, fluorescent microscope or any other device equipped to measure fluorescence.

Within this specification embodiments have been described in a way which enables a dear and concise specification to be written, but it is intended and will be appreciated that embodiments may be variously combined or separated without parting from the invention. For example, the preferred features of the invention can be included in the methods and kits defined herein either separately or in combination with one or more other preferred features.

According to a first aspect, the invention provides a method for evaluating the viability of a parasitic or non-parasitic worm, for example a helminth, comprising
(a) providing a sample comprising one or more parasitic or non-parasitic worms, for example helminths;
(b) contacting the parasitic or non-parasitic worms with a concentration of a first fluorescent dye sufficient to yield a detectable first colour fluorescence in any live parasitic or non-parasitic worms present in the sample and a concentration of a second fluorescent dye sufficient to yield a detectable second different colour fluorescence in any dead parasitic or non-parasitic worms present in the sample; and
(c) detecting the first and second fluorescence of the parasitic or non-parasitic worms.

The method according to the first aspect may further comprise subjecting the the sample of parasitic or non-parasitic worms to an event. In a preferred method, the event is a cell or tissue from another organism, metabolites from other life cycle stages of the test parasitic or non-parasitic worms or metabolites from cells or tissues of other organisms, a serum (including immunoglobulins) or other culture conditions, such as a gas and air mixture.

Preferably, the sample of parasitic or non-parasitic worms is cultured in culture media for at least about 3 hours before subjecting the sample of parasitic or non-parasitic worms to an event. Preferably, the sample is cultured for about 3 hours.

Preferably, the sample of parasitic or non-parasitic worms is cultured in culture media for at least about 24 hours after subjecting the sample of parasitic or non-parasitic worms to an event. Preferably, the sample is cultured for about 24 hours.

According to another aspect, the invention provides a method for screening for the effect of one or more agents on the viability of a parasitic or non-parasitic worm, for example a helminth, comprising
(a) providing a sample comprising one or more parasitic or non-parasitic worms;
(b) contacting the parasitic or non-parasitic worms with one or more test agents;
(c) contacting the parasitic or non-parasitic worms with a concentration of a first fluorescent dye sufficient to yield a detectable first colour fluorescence in any live parasitic or non-parasitic worms present in the sample and a concentration of a second fluorescent dye sufficient to yield a detectable second different colour fluorescence in any dead parasitic or non-parasitic worms present in the sample; and
(d) detecting the first and second fluorescence of the parasitic or non-parasitic worms.

Preferably, the sample of parasitic or non-parasitic worms is cultured in culture media for at least about 3 hours before contacting the parasitic or non-parasitic worms with one or more test agents. Preferably, the sample is cultured for about 3 hours.

Preferably, the sample of parasitic or non-parasitic worms is cultured in culture media for at least about 24 hours after contacting the parasitic or non-parasitic worms with one or more test agents. Preferably, the sample is cultured for about 24 hours.

For example, the test agent could be selected from gambogic acid, sodium salinomycin, ethinyl estradiol, fluoxetidine hydrochloride, bepridil, ciclopirox, miconazole nitrate, chlorpromazine hydrochloride, amphotericin b, niclosamide, rescinnamine, flucytosine, vinblastine, carbidopa, aldicarb, levamisole, ivermectin, praziquantel and auranofin. However, it will be appreciated that any test agent could be used and that the methods of the present invention could be used for high throughput drug screening of compound libraries.

Preferably, the first fluorescent dye is selected from: fluorescein diacetate, calcein AM, newport green(R), Fluo-4AM(R) and Fura-RED(R).

Preferably, the first fluorescent dye is fluorescein diacetate that yields a detectable green fluorescence in any live parasitic or non-parasitic worms present in the sample.

Preferably, the second fluorescent dye is selected from: propidium iodide, ethidium bromide, SYTOX (R) green, SYTOX (R) blue and acridine homodimer.

Preferably, the second fluorescent dye is propidium iodide that yields a detectable red fluorescence in any dead parasitic or non-parasitic worms present in the sample.

Preferably, the methods further comprise allowing sufficient time for fluorescein diacetate and propidium iodide to yield optimal fluorescence.

Preferably, the methods comprise detecting the first and second fluorescences sequentially.

Preferably, the methods comprise detecting the first colour fluorescence before the second colour fluorescence.

Preferably, the first fluorescent dye is fluorescein diacetate and the methods comprise detecting the green fluorescence between about 2 and about 10 minutes or between about 3 and about 12 minutes after contact with the sample containing parasitic or non-parasitic worms. More preferably, the methods comprise detecting the green fluorescence between about 4 and about 8 minutes after contact with the sample containing parasitic or non-parasitic worms, for example about 5 minutes after contact with the sample.

Preferably, the first fluorescent dye is fluorescein diacetate and the methods comprise contacting the parasitic or non-parasitic worms with between about 0.2 μg/ml and about 1.0 μg/ml, preferably about 0.5 μg/ml fluorescein diacetate.

Preferably, the second fluorescent dye is propidium iodide and the methods comprise detecting the red fluorescence about 4 to about 120 minutes after contact with the sample containing parasitic or non-parasitic worms. More preferably, the methods comprise detecting the red fluorescence between about 10 and about 35 minutes, preferably between about 14 and about 29 minutes, for example about 20 minutes after contact with the sample containing parasitic or non-parasitic worms.

Preferably, the second fluorescent dye is propidium iodide and the methods comprise contacting the parasitic or non-parasitic worms with between about 1 μg/ml and about 5 μg/ml, preferably about 2 μg/ml propidium iodide.

Preferably, the methods comprise incubating the parasitic or non-parasitic worms at between about 25 and about 40° C. More preferably, the methods comprise incubating the parasitic or non-parasitic worms at about 37° C.

Preferably, the methods comprise incubating the parasitic or non-parasitic worms in an atmosphere of between about 2% and about 10% CO₂, for example about 5% CO₂. In some cases, incubation of helminths may not require CO₂.

Preferably, the methods further comprise removing the one or more test agents or the event prior to contacting the parasitic or non-parasitic worms with the fluorescent dyes.

Preferably, the methods further comprise providing the parasitic or non-parasitic worms in a culture media, for example DMEM.

In some embodiments, the culture media comprises DMEM lacking phenol red and supplemented with glucose, fetal calf serum (FCS), L-glutamine, penicillin and streptomycin.

Preferably, the methods further comprise removing at least part of the culture media, for example removing FCS, and the one or more test agents or the event prior to contacting the parasitic or non-parasitic worms with the fluorescent dyes.

Preferably, the methods further comprise a wash step prior to contacting the parasitic or non-parasitic worms with the fluorescent dyes to remove at least part of the culture media and any test agent (s) from the sample. The wash step may comprise centrifuging the sample comprising the parasitic or non-parasitic worms, culture media and any one or more test agents. Accordingly, it is preferred that the sample comprises no culture media before contacting the parasitic or non-parasitic worms with the fluorescent dyes.

In other embodiments, no wash step is provided. Accordingly, the methods of the invention may be performed without removing culture media from the sample. Preferably, the culture media has not been supplemented with FCS.

Preferably, the methods further comprise adding an esterase blocker or inhibitor to the sample prior to contacting the parasitic or non-parasitic worms with the fluorescent dyes in a concentration sufficient to block or inhibit esterase present in the culture media and/or in media supplements. Preferably, the esterase inhibitor is a C1 esterase inhibitor.

Preferably, the methods further comprise providing a microtiter or multi-well plate comprising a plurality of wells, each of the wells containing the parasitic or non-parasitic worms. Preferably, the microtiter plate is selected from the group consisting of: a 12-well plate, a 24-well plate, a 96-well plate, a 384-well plate, and a 1536-well plate. Even more preferably, the microtiter plate has 96 wells. In some preferred embodiments, the microtiter plate has 384 wells.

Where a microtiter plate is used, the wells may each contain between 1 and about 15 adult parasitic or non-parasitic worms, more preferably between about 5 and about 10 adult parasitic or non-parasitic worms.

Where a microtiter plate is used, for example a 96 well plate, the wells may each contain between about 100 and about 2500 immature parasitic or non-parasitic worms, for example, schistosomula, more preferably between about 100 and 1000, more preferably between about 500 and about 1000 immature parasitic or non-parasitic worms.

In some embodiments, for example where a 384 well plate is used, the wells may each contain between about 50 and about 500 immature parasitic or non-parasitic worms, for example schistosomula, more preferably between about 100 and about 300 immature parasitic or non-parasitic worms, most preferably about 200 immature parasitic or non-parasitic worms.

Preferably, the means for detecting the fluorescence in the microtitre plate is a multi-well fluorescent plate reader.

Preferably, the means for detecting the fluorescence is selected from: flow cytometry, fluorescent microscopy and fluorescence spectroscopy.

Preferably, the parasitic or non-parasitic worms used in the methods according to the invention are helminths, most preferably of the phylum Platyhelminthes or the phylum Nematoda.

Preferably, the parasitic or non-parasitic worms used in the methods according to the invention are Haemonchus contortus L3 larvae.

Preferably, the parasitic or non-parasitic worms used in the methods are immature adults, preferably immature helminths, preferably immature Platyhelminthes, more preferably S. mansoni schistosomula.

The parasitic or non-parasitic worms may be genetically modified to either extend their life span or promote their death.

Preferably, the methods of the invention are for determining viability of adult male and/or female parasitic or non-parasitic worms, for example helminths.

It will be appreciated that in preferred embodiments of the methods described herein, the parasitic or non-parasitic worms are simultaneously contacted with the first and second dyes.

In preferred embodiments, the methods of the invention can be used to detect phenotypes other than alive or dead. In addition, or alternatively, the methods of the invention can be used to detect metabolic changes. For example, in some embodiments, the methods of the invention are for detecting phenotypic and/or metabolic changes in living parasitic or non-parasitic worms following contact with one or more test agents or after subjecting the sample to an event.

Preferably, the methods are for detecting one or more abnormal phenotypes and/or metabolic changes of living parasitic or non-parasitic worms, for example following contact with one or more test agents or after subjecting the sample to an event.

Preferably, said one or more abnormal phenotypes are selected from rounded shape, hyperactivity, paralysis, flaccid, curled, etc.

Preferably, a change in FTIR spectra from a sample exposed to one or more test agents or an event relative to FTIR spectra from a normal control is indicative of (i) an abnormal phenotype and/or (ii) a metabolic change that does not cause or result from an abnormal phenotype.

Preferably, the normal control is a sample comprising one or more parasitic or non-parasitic worms, for example helminths, known to have a normal phenotype (i.e. known not to show an abnormal phenotype, for example selected from rounded shape, hyperactivity, paralysis, flaccid, curled, etc.) and/or known to have a normal metabolic profile (i.e. known not to show a metabolic change).

Preferably, the nature of the abnormal phenotype and/or metabolic change is determined by comparing the I- IR spectra produced from the sample with one or more reference FI IR spectra indicative of abnormal phenotypes and/or metabolic changes.

For example, in one embodiment parasitic or non-parasitic worms are cultured with control compounds known to induce a phenotypic and/or metabolic change (as described elsewhere herein) in either 96- or 384-well microtiter plates. Other wells are cultured with test compounds, where the phenotype and/or metabolic change (if any) is unknown. After 24 hrs, the culture media is collected and transferred to 96- or 384-well silica plates by a multichannel pipette. Supernatants are dried and then placed into a FTIR spectometer where they are analyzed in the infrared spectra. FTIR spectra from supernatants of test compound treated parasitic or non-parasitic worms that segregate with FTIR spectra from supernatants of control compound treated parasitic or non-parasitic worms (inducing a discernable phenotype and/or metabolic profile) is selected as being ‘of interest’.

Preferably, the FTIR spectra are analysed using principal component analysis (PCA).

According to one aspect of the present invention, there is provided a method for detecting phenotypic and/or metabolic changes in a sample of living parasitic or non-parasitic worms, for example helminths following contact with one or more test agents or after subjecting the sample to an event, the method comprising
(a) providing a sample comprising one or more parasitic or non-parasitic worms, for example helminths;
(b) contacting the parasitic or non-parasitic worms with one or more test agents or subjecting the sample to an event;
(c) performing spectral analysis of the sample; and
(d) comparing the spectra produced from the sample with an FTIR spectra from one or more reference standards indicative of abnormal phenotype and/or metabolic change to identify the presence of one or more abnormal phenotypes and/or metabolic changes in the sample.

Preferably, the spectra produced from the sample and the spectra from the one or more reference standards are compared using principal component analysis and/or discriminate function analysis (DFA).

Preferably, FTIR spectral analysis is performed on the supernatant of the sample comprising one or more parasitic or non-parasitic worms.

Preferably, FTIR spectral analysis is performed on worm bodies of the sample comprising one or more parasitic or non-parasitic worms.

According to another aspect, the invention provides a kit for evaluating the viability of parasitic or non-parasitic worms, for example helminths or for screening for the activity of one or more agents or an event on parasitic or non-parasitic worm, for example helminth viability, the kit comprising:

a microtiter plate comprising a plurality of wells;

a culture media;

a first fluorescent dye that yields a detectable first colour fluorescence in any live parasitic or non-parasitic worms; and

a second fluorescent dye that yields a detectable second colour fluorescence in any dead parasitic or non-parasitic worms.

According to further aspect, the invention provides a kit for evaluating phenotypic and/or metabolic changes of parasitic or non-parasitic worms, for example helminths following contact with one or more test agents or after subjecting a sample of parasitic or non-parasitic worms, for example helminths to an event, the kit comprising:

a microtiter plate comprising a plurality of wells;

a culture media; and

instructions for performing FTIR analysis.

Preferably, the kit further comprises a wash medium. Preferably, the wash medium is lyophilised or in liquid form.

Preferably, the kit further comprises a positive control anthelminth agent selected from: auronofan, ethanol, phenol, iodine, methanol, formaldehyde, dimethyl sulphate, hydrogen peroxide, hydrochloric acid, acetic acid, formic acid and sodium dodedecyl sulphate.

Preferably, the first fluorescent dye in the kit is selected from: fluorescein diacetate, calcein AM, Newport green, Fluo-4AM(R) and Fura-RED(R).

Preferably, the second fluorescent dye in the kit is selected from: propidium iodide, ethidium bromide, SYTOX(R) Green, SYTOX(R) Blue and acridine homodimer.

Preferably, the culture media in the kit is lyophilised.

Preferably, the kit further includes a plurality of parasitic or non-parasitic worms, for example helminths. Preferably, the plurality of parasitic or non-parasitic worms in the kit are disposed such that each of a plurality of the wells contains one or more of the parasitic or non-parasitic worms.

Preferably, the kit further comprises instructions to the use of the kit for high-throughput screening for the effect of one or more test agents or an event on a parasitic or non-parasitic worm, for example a platyhelminth or nematode.

Preferred embodiments of this invention are described herein, including the best mode known to the inventors for carrying out the invention. It should be understood that the illustrated embodiments are exemplary only, and should not be taken as limiting the scope of the invention.

### DETAILED DESCRIPTION OF THE INVENTION AND PREFERRED EMBODIMENTS

With only one effective drug, praziquantel, currently used to treat most worldwide cases of schistosomiasis there exists a pressing need to identify alternative anthelmintics before the development of praziquantel-resistant schistosomes removes our ability to combat this neglected tropical disease. At present, the most widely adopted methodology used to identify promising new anti-schistosome compounds relies on time consuming and subjective microscopic examination of parasite viability in response to in vitro schistosome/compound co-culturing. In our continued effort to identify novel drug and vaccine targets, we detail a dual-fluorescence bioassay that can objectively be used for assessing Schistosoma mansoni schistosomula viability in a medium or high- throughput manner to suit either academic or industrial settings. The described methodology replaces subjectivity with sensitivity and provides an enabling technology useful for rapid in vitro screens of both natural and synthetic compound libraries. It is expected that results obtained from these quantifiable in vitro screens would prioritize the most effective anti-schistosomal compounds for follow-up in vivo experimentation. This highly-adaptable dual-fluorescence bioassay could be integrated with other methods for measuring schistosome phenotype and, together, be used to greatly accelerate our search for novel anthelmintics.

This single property (simultaneous detection of both PI and FDA measures) has allowed us to develop a fluorescence-based, microtiter-plate bioassay to improve detection of schistosome viability, which is high-throughput (96- and 384-well capacity), quantitative and provides objective readouts (fluorescence intensity units) of parasite survival during in vitro culture.

Using this flexible bioassay, we demonstrate its versatility in detecting schistosome survival in response to thioredoxin glutathione reductase (TGR) inhibition (Kuntz A N, Davioud-Charvet E, Sayed A A, Calif L L, Dessolin J, et al. (2007) Thioredoxin Glutathione Reductase from Schistosoma mansoni: An Essential Parasite Enzyme and a Key Drug Target. PLoS Medicine 4: e206). Furthermore, we adopt this assay to provide quantitative estimates of schistosome viability in the presence of recently identified small compounds with previously described (Abdulla M H, Ruelas D S, Wolff B, Snedecor J, Lim K C, et al. (2009) Drug Discovery for Schistosomiasis: Hit and Lead Compounds Identified in a Library of Known Drugs by Medium-Throughput Phenotypic Screening. PLoS Neglected Tropical Diseases 3: e478) and unknown (Berriman M, Haas B J, LoVerde P T, Wilson R A, Dillon G P, et al. (2009) The genome of the blood fluke Schistosoma mansoni. Nature 460: 352-358) chemotherapeutic activities. Implementation of this novel screening platform by academia and industrial stakeholders will enable inter-laboratory comparisons of in vitro parasite manipulations to be routinely and quickly performed, vastly accelerating the search for novel anti-schistosomal lead targets.

Methods

Schistosomula Preparation and Culturing

Schistosoma mansoni (Puerto Rican strain) infected Biomphalaria glabrata snails were provided by Fred Lewis (Biomedical Research Institute, Rockville, Md., USA). Cercariae were shed from infected snails by exposure to light (60 min at room temperature, RT) and subsequently converted to schistosomula by mechanical transformation (Colley D G, Wikel S K (1974) Schistosoma mansoni: simplified method for the production of schistosomules. Experimental Parasitology 35: 44-51). Schistosomula were purified away from cercarial tails by centrifugation through a 60% percoll gradient (Lazdins J K, Stein M J, David J R, Sher A (1982) Schistosoma mansoni: rapid isolation and purification of schistosomula of different developmental stages by centrifugation on discontinuous density gradients of Percoli. Experimental Parasitology 53: 39).

Microscope examination was used to assess the quantity and quality of purified schistosomula. Schistosomula were cultured at 37° C. in T25 tissue culture flasks containing 9 ml DMEM (Dulbecco's Modified Eagle Medium, Sigma-Aldrich), lacking phenol red but containing 4500 mg/I glucose, supplemented with 10% foetal calf serum, 2 mM L-glutamine, 200 U/ml penicillin, 200 μg/ml streptomycin (all Sigma-Aldrich) in an atmosphere of 5% CO₂ for 24 hr before any further experimental manipulations proceeded. Negligible parasite death occurred in this media during the 24 hr culturing period. Following this, schistosomula were aliquoted into black-sided, flat-bottom (optically clear), 96-well microtiter plates (Fisher Scientific) in 200 μl media or black-sided, flat-bottom (optically clear), 384-well microtiter plates (Matrix) in 40 μl media.

Heat killed schistosomula were also prepared by incubating the 24 hr cultivated parasites at 65° C. for 10 min. These dead schistosomula were allowed to cool to 37° C. before being used in subsequent experiments.

Microscopy

Live and heat killed schistosomula stained with optimal concentrations (empirically derived from Jones K, Senft J (1985) An improved method to determine cell viability by simultaneous staining with fluorescein diacetate-propidium iodide. The Journal of Histochemistry and Cytochemistry 33: 77-79) of propidium iodide (PI, 2.0 μg/ml; Sigma-Aldrich), fluorescein diacetate (FDA, 0.5 μg/ml; Sigma-Aldrich) or both fluorophores were visualized at ×100 magnification using a Leica Axioplan microscope equipped with FITC (494 excitation) and Rhodamine (536 excitation) filters and a mercury vapor light source. A Hamamatsu CA74295 camera with Wasabi Version 1.4 software was used to capture photographic images of stained schistosomula.

Schistosomula, co-incubated with test compounds, were fluorescently visualized as above or unstained at ×100 magnification using an Olympus CK2 inverted microscope equipped with a stage extension plate and specimen holder for handling microtiter plates. A Kodak EasyShare DX7440 digital camera was used to capture images of unstained schistosomula.

Compound Storage, Handling and Schistosomula Screening

All compounds were purchased from Sigma-Aldrich and included; gambogic acid, sodium salinomycin, ethinyl estradiol, fluoxetidine hydrochloride, bepridil, ciclopirox, miconazole nitrate, chlorpromazine hydrochloride, amphotericin b, niclosamide, rescinnamine, flucytosine, vinblastine, carbidopa, praziquantel, ivermectin, levamisole, aldicarb and auranofin.

Stock solutions of all compounds were made up at 1 mM in appropriate solvents and stored at −80° C. All compounds were added to black-sided, flat-bottom (optically clear), 96-well microtiter plates containing schistosomula (1000 parasites/well in triplicate) at 10 μM concentrations. Schistosomula were cultured (as already indicated; 37° C., 5% CO₂) in the presence of each compound for 24 hr before viability levels were assessed.

Schistosomula Viability Determination in Response to Test Compounds

After the 24 hr culturing period in the presence of test compounds, and prior to addition of fluorescent dyes, all schistosomula were washed three times to remove test compound and culture media supplements. Each wash consisted of centrifuging microtiter plates containing schistosomula at 100×g for 5 min, removal of half the old culture media and replacement with an equal quantity of fresh DMEM (lacking phenol red). After washing the parasites, PT and FDA were simultaneously added to each well of the microtiter plate to obtain a final concentration of 2.0 μg/ml and 0.5 μg/ml respectively.

The 96-well microtiter plates, now containing fluorescently labeled parasites, were subsequently loaded into a BMG Labtech Polarstar Omega plate reader containing appropriate filters for the simultaneous detection of PI (544 nm excitation/620 nm emission) and FDA (485 nm excitation/520 nm emission). All fluorescent values were obtained with the plate reader incubator set at 37° C. to ensure efficient esterase conversion of FDA to fluorescein within live schistosomula. The plate reader automatically sets the photo multiplier tube (PMT) gain for each fluorescent dye and this may slightly vary between experiments. Inclusion of appropriate control samples (live and dead schistosomula) compensates for any inter-plate variations in gain settings.

Data Handling and Statistical Analysis

All data were exported into Microsoft Excel for organization and into Minitab (Version 14) for statistical analyses. A One-Way Analysis of Variance (ANOVA) followed by post hoc testing with Fisher's least significant difference (LSD) was used to detect statistical differences between treatments. Viability percentages were either: (1) converted into probits for auranofin dose response curve generation and calculation of LD₅₀ values or (2) Arcsin transformed in order to stabilize variances prior to the use of appropriate statistical analyses.

Numbers of live and dead schistosomula in each well of a microtiter plate were calculated using the following equations:

$$Live ( F D A fluorescence ) = \frac{Sample - negative control}{Positive control - negative control}$$

$$Dead ( PI fluorescence ) = \frac{Sample - media control}{Negative control - media control}$$

‘Samples’ represent fluorescence intensity units collected from parasites incubated with test compounds. ‘Negative control’ represents fluorescence intensity units collected from parasites killed with 10 μM auranofin or heat shock (10 min incubation at 65° C.), while the ‘Positive control’ represents fluorescence intensity units collected from untreated parasites. ‘Media control’ represents fluorescence intensity units originating from wells containing only media (no parasites).

To determine schistosomula viability in response to each test compound, a second calculation was employed. This normalization compensated for inter-well variability in schistosomula numbers across the microtiter plate, and to facilitate accurate viability comparisons between test compounds from different microtiter plates:

$$\% Viability = \frac{Live ( F D A fluorescence )}{Dead ( PI fluorescence ) + Live ( F D A fluorescence )} \times 100$$

Results

Microscope Examination of In Vitro Transformed Schistosomula

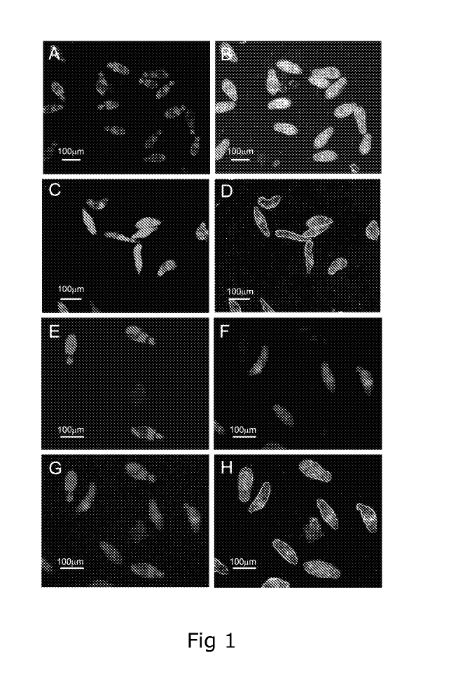

To first determine whether propidium iodide (PI) and fluorescein diacetate (FDA) could be used individually to detect dead or live in vitro transformed S. mansoni schistosomula, an initial fluorophore uptake experiment was performed with parasite viability assessed by fluorescent microscopy. Here, in vitro transformed schistosomula, cultured for 24 hr, were either heat killed at 65° C. for 10 min or left undisturbed at physiological conditions (37° C.). Heat-killed parasites were then subsequently single-stained with PI (2.0 μg/ml) whereas live parasites were incubated with FDA (0.5 μg/ml). All dead parasites fluoresced (FIG. 1A, 16 out of 16) when visualized for PI uptake at 536 nm, with corresponding polarized bright field microscopy imaging of the same schistosomula samples providing morphological confirmation of death (i.e. parasites that were uniform in shape and size and displayed no movement (FIG. 1B)). Furthermore, all live parasites fluoresced (FIG. 1C, 10 out of 10) when visualized for FDA uptake at 494 nm, with corresponding polarized bright field microscopy imaging of the same schistosomula samples providing visual confirmation of a physiological normal phenotype (i.e. parasites displaying a variety of shapes and sizes as the result of movement during in vitro culturing, FIG. 1D). Further quantification of fluorophore uptake was performed on four different regions of slides containing PI stained schistosomula and from five different regions of slides containing FDA stained schistosomula. Here 100% of the parasites were stained with either of the two examined fluorophores (39 out of 39 dead schistosomula were PI positive and 48 out of 48 live schistosomula were FDA positive), confirming the utility of the chosen fluorophores for evenly staining schistosomula (data not shown).

By demonstrating that PI could effectively be used to stain dead schistosomula and FDA was capable of identifying live parasites, a further experiment was conducted to determine whether PI and FDA could simultaneously be used to detect individual live (FDA positive) and dead (PI positive) in vitro transformed schistosomula from a mixed population of differentially viable parasites. Here, in vitro transformed schistosomula, cultured for 24 hr, were either heat killed at 65° C. for 10 min or left undisturbed at physiological conditions (37° C.). Equal numbers of live and dead schistosomula were mixed, simultaneously stained with PI (2.0 μg/ml) and FDA (0.5 μg/ml) and examined for the presence or absence of differential fluorophore emission. Whereas heat-killed schistosomula exhibited strong PI staining (minimal FDA staining), physiologically normal schistosomula conversely displayed strong FDA staining (no observable PI staining) (compare FIG. 1E and FIG. 1F). This differential staining of individual live and dead parasites was further supported by observations of undetectable co-localization of PI and FDA signals (within the same cell of an individual schistosomulum, detectable as a yellow signal), schistosomula phenotype and parasite motility as detected by both fluorescent and polarized bright field microscopy (FIGS. 1G and 1H).

Optimization of Fluorophore/Schistosomula Incubation Time

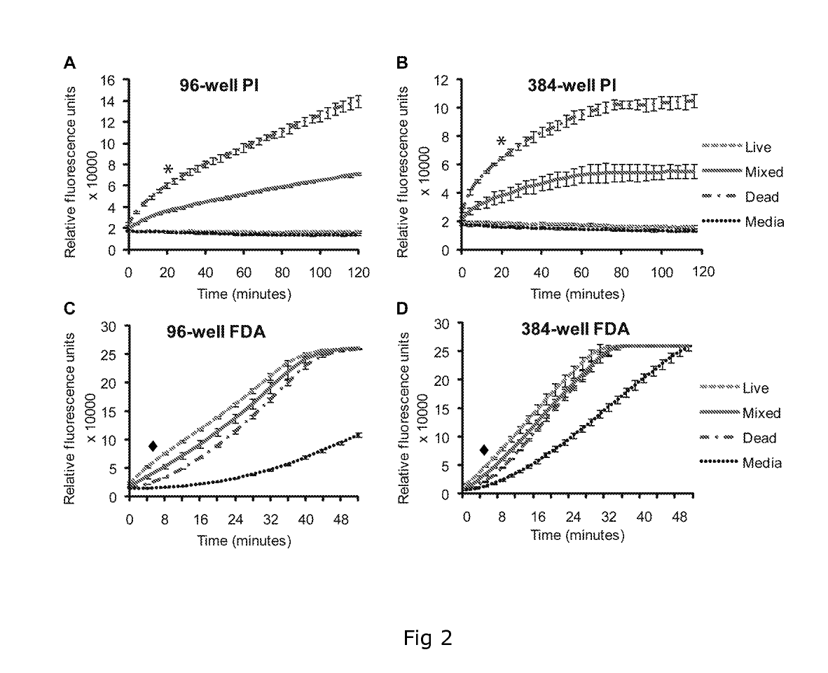

A critical first parameter in translating these observations of differential PI and FDA staining of schistosomula to a method that takes advantage of the high-throughput potential of a microtiter plate assay (in both 96- and 384-well formats) was to determine the optimal timeframe that each fluorescent dye should be incubated with parasite samples to insure maximal reproducible detection of viability (FIG. 2). Here, in vitro transformed schistosomula, cultured for 24 hr, were either killed by heat shock (65° C. for 10 min) or left unaltered at physiological culturing conditions (37° C.). Three samples were subsequently derived from these schistosomula cultures, which included physiologically normal, untreated parasites, heat-killed parasites and an equal mixture of physiologically normal and heat-killed parasites. Derived schistosomula samples (in triplicate) were co-stained with PI (2.0 μg/ml) and FDA (0.5 μg/ml) and subjected to fluorescent intensity measurements (BMG Labtech Polarstar plate reader) every minute for 120 min. Fluorescent intensity values derived from triplicate wells containing DMEM (lacking phenol red), PI (2.0 μg/ml) and FDA (0.5 μg/ml) were also obtained over this timeframe (FIG. 2, dotted line).

Throughout the assayed 120 min timeframe, the three tested schistosomula samples (regardless of plate format) demonstrated clear differences in PI fluorescence emission, with the dead parasites demonstrating the highest PI values, the live parasites demonstrating the lowest PI values and the mixed population of live and dead parasites demonstrating intermediate PI values between the extremes (FIGS. 2A and 2B). While statistical significance in measured PI fluorescent emission continued to increase (i.e. p<0.05 at 1 min and p<0.001 for any time point after 4 min) with time for any of the compared schistosomula samples, PI fluorescent emission differences between physiologically normal schistosomula (live) and DMEM (lacking phenol red) samples remained small throughout the duration of the 120-minute timeframe and never reached statistical significance (p<0.001). Therefore, based on these analyses, the optimal PI incubation time to distinguish between dead and live schistosomula is between 4 and 120 min, regardless of plate format. We chose 20 min to collect PI data as it provided an adequate time window to process multiple microtiter plates (indicated in FIGS. 2A and 2B) and was well within the calculated window of accurately being able to determine statistical significance amongst live and dead schistosomula.

Whereas PI staining of parasite samples yielded differential fluorescent results over the entire 120-minute timeframe (FIGS. 2A and 2B), FDA staining of live, dead and mixed schistosomula populations in either 96-well or 384-well microtiter plate formats generated fluorescent data that quickly reached a plateau (51 min for 96-well microtiter plates, FIG. 2C and 32 min for 384-well microtiter plates, FIG. 2D). Therefore, collection of FDA fluorescence was halted at 51 min. Nonetheless, FDA fluorescent intensity units measured across these time intervals produced data as expected with live parasites fluorescing brightest, dead parasites weakest and mixed live/dead parasite populations intermediate between the two (FIGS. 2C and 2D). Furthermore, differences in detected FDA fluorescence between any of the three schistosomula samples (in both microtiter plate formats) were found to be statistically significant between 3 min and 12 min. However, unlike the PI timecourse (FIGS. 2A and 2B), differences in FDA fluorescent emission originating from wells containing DMEM and all schistosomula samples were statistically significant beginning at 3 minutes (p<0.05). This finding makes DMEM unsuitable for use as a blank for detecting non-specific FDA fluorescence. Therefore, based on these analyses, the optimal FDA incubation time to distinguish between dead and live schistosomula in both microtiter plate formats is between 3 and 12 min. We chose 5 min (indicated in FIG. 2C and 2D) to collect FDA data in both microtiter plate formats as this fluorophore is prone to spontaneous hydrolysis (Clarke J M, Gillings M R, Atlavilla N, Beattie A J (2001) Potential problems with fluorescein diacetate assays of cell viability when testing natural products of antimicrobial activity. Journal of Microbiological Methods 46: 261-267).

Determination of Assay Sensitivity

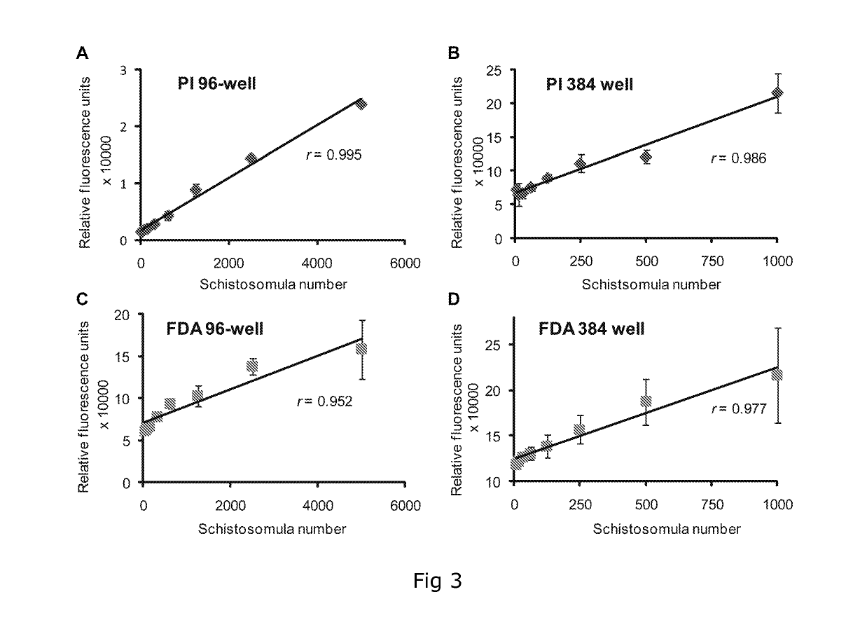

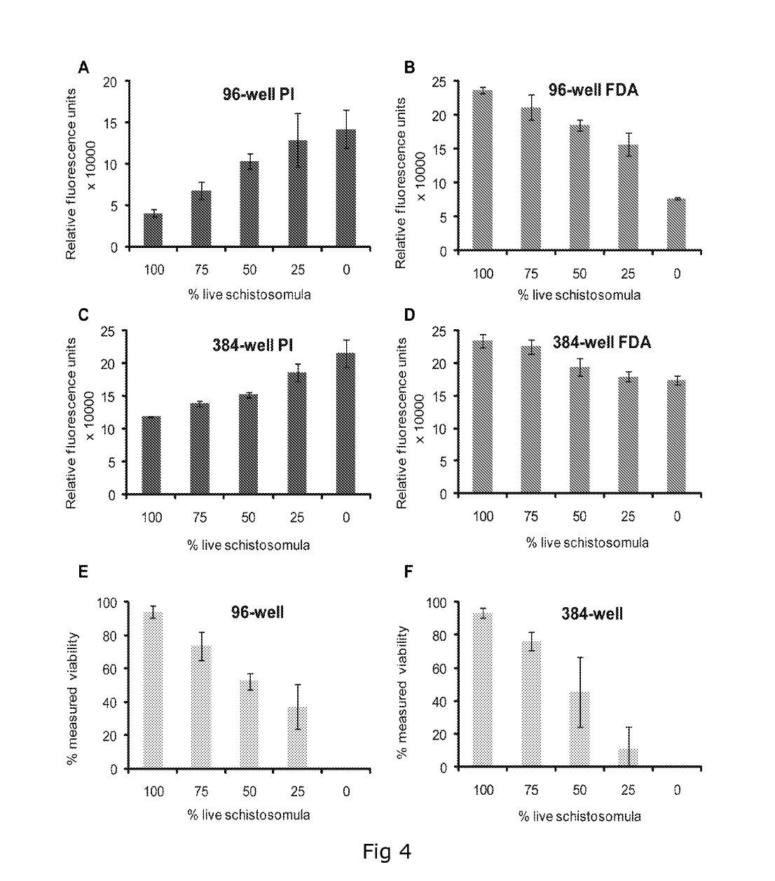

To determine the sensitivity limits of this fluorescence-based assay in measuring schistosomula viability in a medium and a high throughput manner, two different experimental approaches were considered (FIGS. 3 and 4). In the first approach (FIG. 3A-D), schistosomula were serially diluted (5000 reducing to 36 parasites for a medium throughput 96-well plate format; 1000 reducing to 8 parasites for a high-throughput 384-well plate format, in triplicate) to identify the absolute minimum number of parasites that could be reproducibly detected. Briefly, in vitro transformed schistosomula, cultured for 24 hr, were either heat killed (65° C. for 10 min) or left unaltered at physiological conditions (37° C.). After serially diluting these differentially treated schistosomula samples, FDA or PI fluorophores were added and measurement of fluorescence intensity proceeded, within the parameters determined above (5 min for FDA and 20 min for PT).

Correlation analysis revealed a strong linear relationship between PI fluorescence and dead schistosomula number. For the 96-well plate format (FIG. 3A) a high correlation (r=0.995) existed between the measured PT fluorescence and numbers of dead schistosomula sampled, while for the 384-well plates (FIG. 3B) a slightly lower correlation (r=0.986) existed between measured PI fluorescence and numbers of dead schistosomula.

Correlation analysis of FDA fluorescence and live schistosomula also revealed a strong linear relationship (r=0.952 for the 96-well plate format, FIG. 3C and r=0.977 for the 384-well format, FIG. 3D), although the linearity was affected by an FDA-associated fluorescence plateau effect seen when increased numbers of schistosomula were assayed. From these experiments, it was determined that optimal numbers of schistosomula for assays conducted in a 96-well microtiter plate are between 500 and 2500 parasites per well, while for assays conducted in a 384-well microtiter plate, optimal numbers of schistosomula are between 50 and 500 per well.

The second approach used to interrogate the sensitivity of this fluorescence-based assay for detecting schistosomula viability was to replicate conditions where varying percentages of live and dead schistosomula would be found in the same sample (i.e. in vitro drug assays, where the drug tested is less than 100% efficient in killing). By mixing different percentages of live (physiologically normal) and dead (heat killed) parasites (FIG. 4), we tested the ability of PI (FIGS. 4A and 4C) and FDA (FIGS. 4B and 4D) to distinguish viability in a population of 1000 schistosomula in a 96-well plate (FIG. 4E) and 200 schistosomula in a 384-well plate (FIG. 4F). In both plate formats, intra-well PI fluorescence decreased and FDA fluorescence increased when greater percentages of live schistosomula were being examined. This made it possible to differentially identify schistosomula viability in intervals of 25% for both 96- and 384-well plate formats. However, the standard deviations reported for some values indicated that there was variability inherent in pipetting with discrete units of this size. The use of a dual staining method was, therefore, important for counteracting any pipetting errors and verifying that differences in fluorescence are genuinely caused by differences in mortality.

The observation that calculated, intra-well schistosomula viability (mathematically derived from plate reader measurements of both PI and FDA, FIG. 4A-D) paralleled the proportion of viable schistosomula dispensed into each well (FIGS. 4E and 4F), demonstrated that FDA and PI could cooperatively be used to sensitively and accurately differentiate amongst percentages of viable parasite populations within either 96- or 384-well microtiter plate formats. This is essential as any high-throughput anthelmintics screening assay may include compounds that do not induce 100% schistosomula lethality and, therefore, use of both PT and FDA allow for accurate quantification across a range of viability endpoints (between 0% viable to 100% viable).

Validation of the Dual Fluorescent Assay for Identifying Therapeutic Lead Compounds

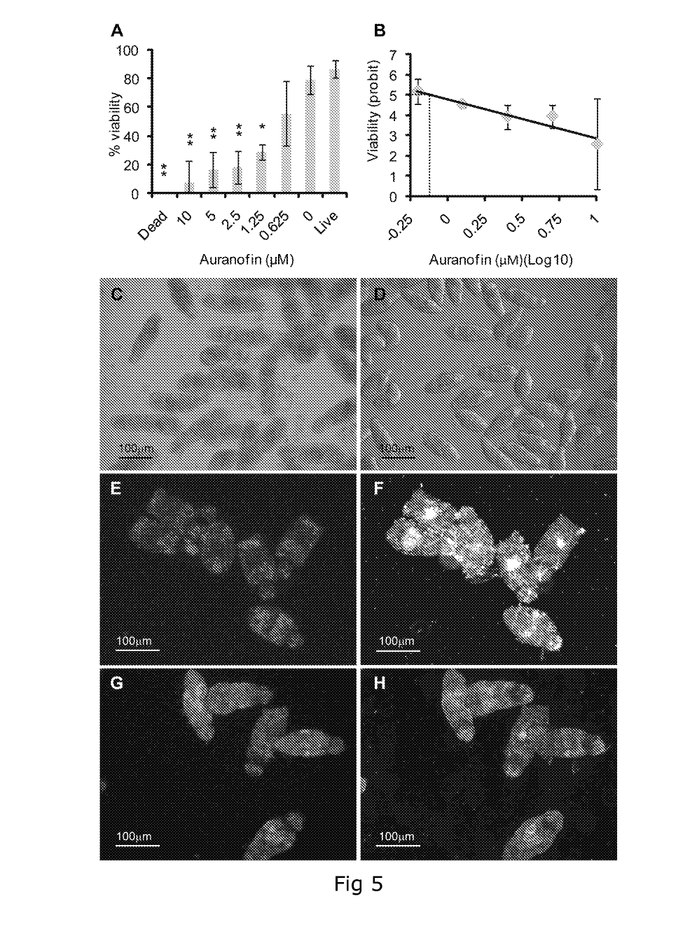

To validate this fluorescent-based viability assay for practical application in medium to high-throughput drug screening, it was used to assess the efficacy of a known anti-schistosomula compound, auranofin (FIG. 5). An inhibitor of parasite thioredoxin glutathione reductase (TGR), auranofin has been shown (estimated by light microscopy examination) to be 100% lethal to in vitro cultured, mechanically-transformed schistosomula at 10 μM concentrations, 24 hr after treatment (Kuntz A N, Davioud-Charvet E, Sayed A A, Calif L L, Dessolin J, et al. (2007) Thioredoxin Glutathione Reductase from Schistosoma mansoni: An Essential Parasite Enzyme and a Key Drug Target. PLoS Medicine 4: e206).

In agreement with published reports, there is a clear and titratable anti-schistosomula effect mediated by auranofin with the dual fluorescent staining procedure allowing viability quantification of each drug concentration at 24 hours post-treatment (FIG. 5A). Percent viability transformations into probit values also allowed an auranofin LD₅₀ of 0.82±0.49 to be calculated (FIG. 5B). Maximum drug effect was seen at 10 μM, where microscopic examination of schistosomula confirmed that death was 100% (FIGS. 5C, 5E and 5F), when compared to untreated parasites (FIG. 5D). At an auranofin concentration within the calculated LD₅₀ range (1 μM), schistosomula contained cells either labeled with PI or FDA throughout the lophotrochozoan body (FIGS. 5G and 5H). Statistically-significant, auranofin-mediated mortality, compared to either vehicle treated (DMSO) schistosomula or untreated parasites (live), was also observed for drug concentrations of 5 μM, 2.5 μM and 1.25 μM.

While the methods described herein confirmed that auranofin does induce schistosomula death (FIG. 5), the results additionally demonstrated a much finer sensitivity in detecting auranofin LD₅₀ levels (concentration of auranofin that kills 50% of the assayed biological material). Kuntz et al. (Kuntz A N, Davioud-Charvet E, Sayed A A, Califf L L, Dessolin J, et al. (2007) Thioredoxin Glutathione Reductase from Schistosoma mansoni: An Essential Parasite Enzyme and a Key Drug Target. PLoS Medicine 4: e206), using microscopy, reported LD₅₀ levels of auranofin on schistosomula to be between 2 μM and 5 μM. Our auranofin titration series, detected by fluorescent-based microtiter plate quantification, demonstrated that this compound has an anti-schistosomula LD₅₀ activity of 0.82 μM±0.49 μM. This objective determination is approximately 4 fold more sensitive than what was previously published and clearly demonstrates the increased sensitivity of the methods described herein and a standardized methodology. Interestingly, schistosomula, treated with 1 μM auranofin (within the calculated LD₅₀ range) and visualized by epifluorescent microscopy, displayed cells that were stained with either PI or FDA throughout the body (FIG. 5G). This finding clearly indicates that the methods are sensitive enough to detect compound-induced changes in fluorescence affecting different cell populations within individual schistosomula as well as between schistosomula and may be useful in identifying compounds that induce cellular stress (in addition to whole organism viability).

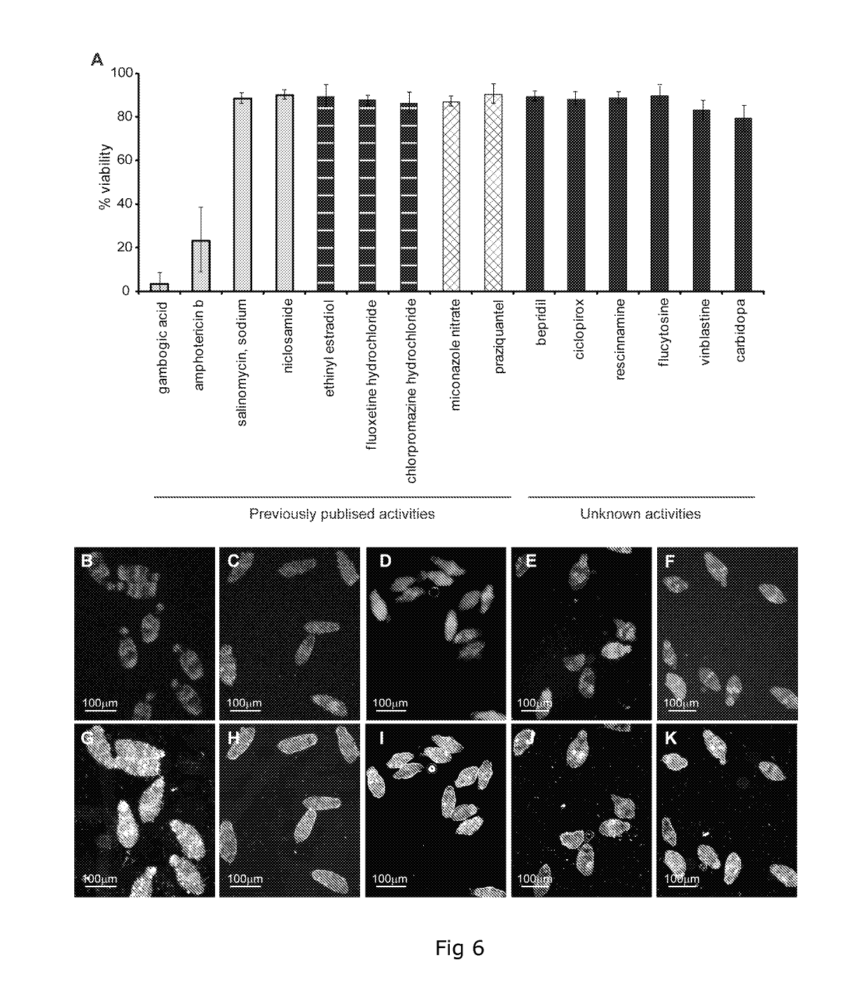

Further validation of this viability assay was next performed by assessing the effect of compounds with previously-identified (Abdulla M H, Ruelas D S, Wolff B, Snedecor J, Lim K C, et al. (2009) Drug Discovery for Schistosomiasis: Hit and Lead Compounds Identified in a Library of Known Drugs by Medium-Throughput Phenotypic Screening. PLoS Neglected Tropical Diseases 3: e478) or suggested anti-schistosomal activities (Berriman M, Haas B J, LoVerde P T, Wilson R A, Dillon G P, et al. (2009) The genome of the blood fluke Schistosoma mansoni. Nature 460: 352-358) (FIG. 6). These compounds included four (gambogic acid, sodium salinomycin, niclosamide nitrate and amphotericin b) that have been described to induce schistosomula mortality, three (ethinyl estradiol, fluoxetine hydrochloride and chlorpromazine hydrochloride) that have been reported to induce schistosomula over-activity, two (miconazole nitrate and praziquantel) that have been shown to produce a shape alteration (‘rounded’ phenotype) and six (bepridil, ciclopirox, rescinnamine, flucytosine, vinblastine and carbidopa) that have never been tested on schistosomula. Of the tested compounds with previously-recorded effects on schistosome phenotypes, our dual-fluorescent viability screen, reassuringly showed broad agreement (FIG. 6A). Here, the compounds previously described as inducing an over-active (ethinyl estradiol, fluoxetine hydrochloride and chlorpromazine hydrochloride) or rounded (miconazole nitrate and praziquantel) phenotype, as expected, did not affect schistosomula viability. Fluorescent readings obtained from wells containing parasites treated with these compounds were no different from wells containing untreated parasites (average viability 84.3%, data not shown). Microscopic examination of these treated parasites confirmed their viability (e.g. FIG. 6E). However, only two of four previously defined anti-schistosomula compounds (gambogic acid and amphotericin b) induced measurable death as determined by our dual-fluorescent viability assay (confirmed by microscopic examination of schistosomula, e.g. FIG. 6B). Sodium salinomycin and niclosamide produced fluorescent viability measurements similar to those derived from untreated parasites. Detailed microscopic examination of schistosomula treated with sodium salinomycin demonstrated that the parasites were strongly FDA positive (with some PI positive cells) (FIG. 6C), dark and granular (FIG. 6H), but motile whereas niclosamide treated schistosomula appeared morphologically (FIG. 6I) and fluorescently (FIG. 6D) similar to praziquantel treated parasites (rounded phenotype, FIG. 6E, FIG. 6J). Of the six compounds with an unknown, but suggested, anti-schistosome effect, none showed strong decreases in viability as determined by the dual fluorescence assay or epifluorescent microscopy (e.g. ciclopirox, FIG. 6F) under the conditions tested in this study. However, upon further microscopic examination, some compounds did induce shape alterations in schistosomula phenotype (e.g. ciclopirox, FIG. 6K).

The comparison of the results obtained herein with the subjective methods of the prior art emphasizes the potential for unintentional misclassification of schistosomula viability when solely reliant upon subjective microscopic examination of parasite phenotype. Without application of objective measures, errors in false positive discovery can contaminate a high-throughput dataset and lead to incorrect leads being pursued, wasting valuable time and resources. The objective measures used here, differential schistosomula uptake of two fluorophores and corresponding microtiter plate reader detection of fluorescence emission, additionally include an important built-in element of self-verification (individual parasites will stain brightly with one fluorophore and weakly for the second fluorophore). This characteristic insures that each schistosomula assay will provide two independent, quantitative measures of compound efficacy, which adds a level of confirmation difficult to achieve with microscopic measurements.

Schistosomula 384-Well Data

All the original experiments were run in 96-well plates with approximately 1000 schistosomula per well. In an effort to reduce the number of parasites for each experiment, the use of 384-well plates was investigated.

A titration series of schistosomula was run initially to investigate the effect of parasite number on fluorescence. As shown in FIGS. 2, 3 and 4, the results were similar to what was seen for a 96-well plate.

Adult Worm Data Both 96- and 384-Well

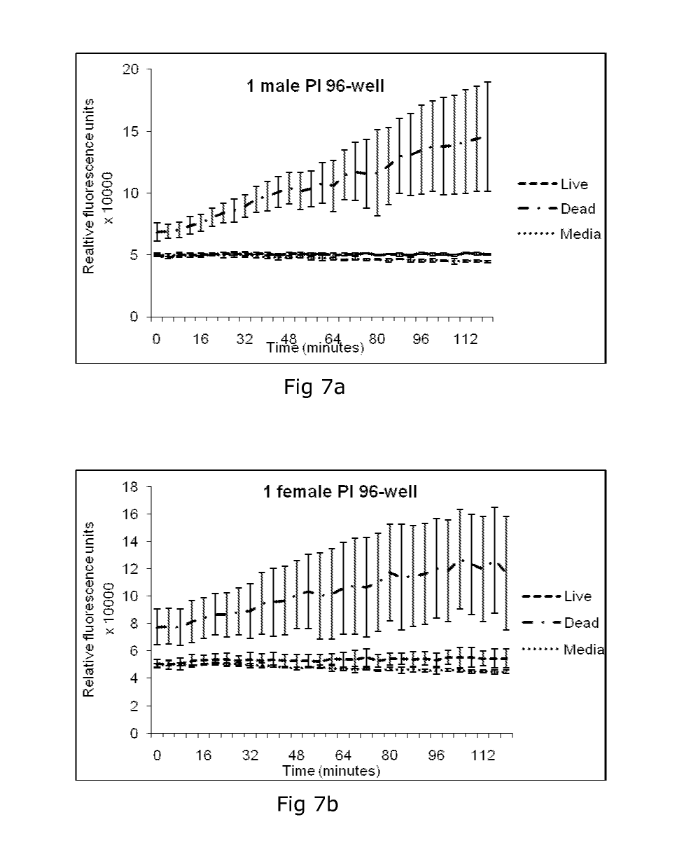

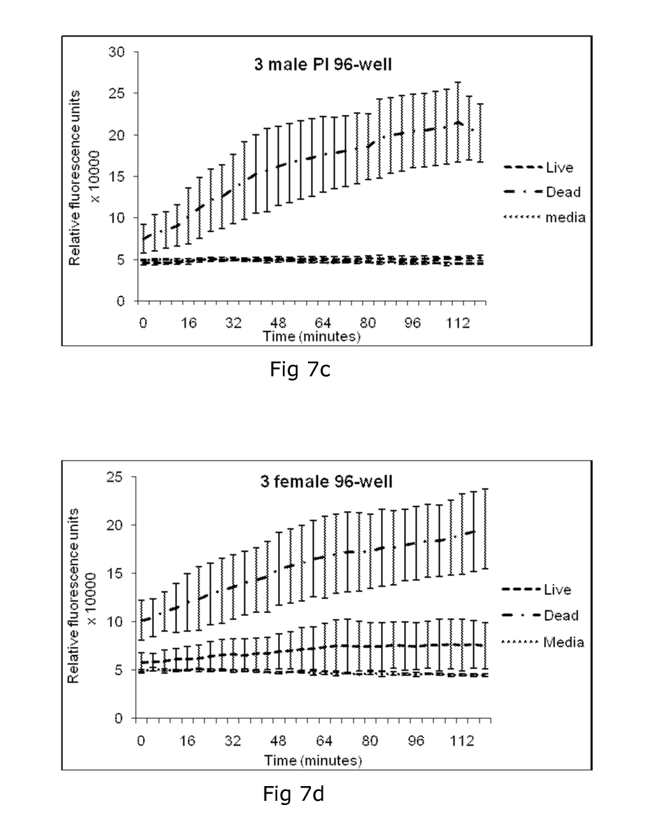

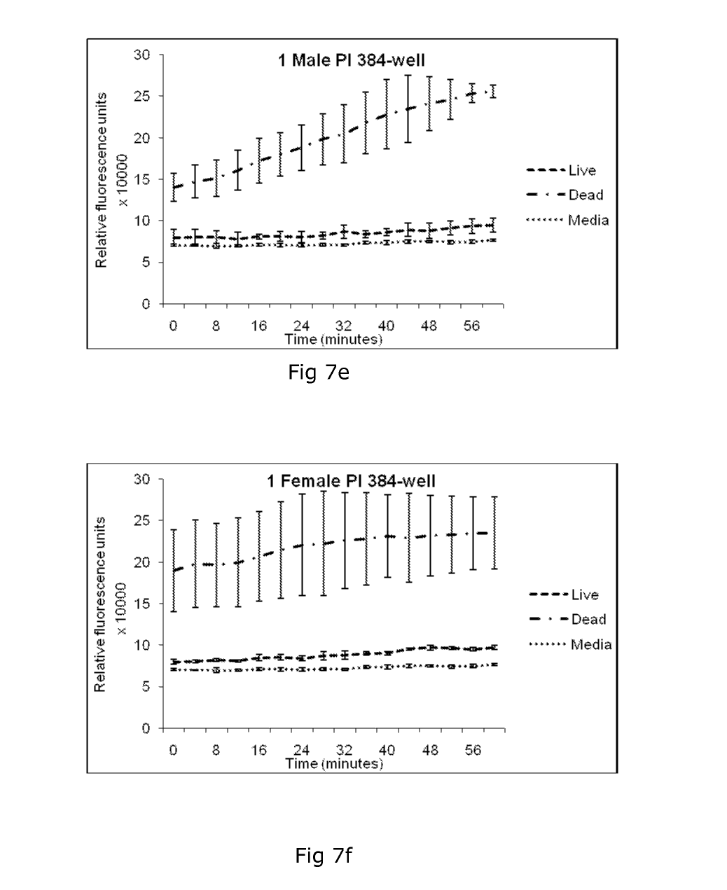

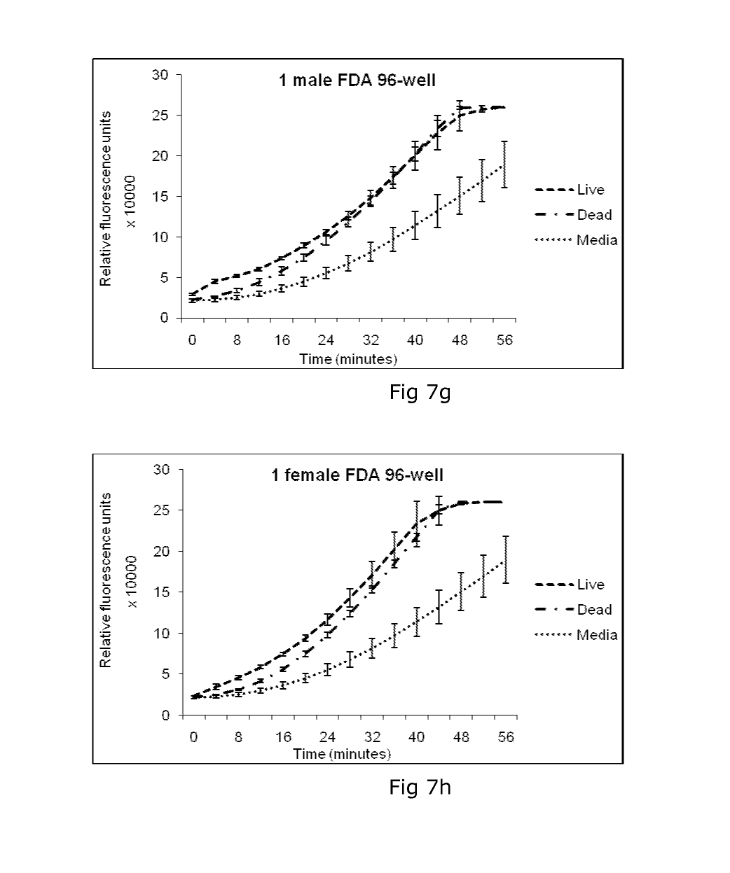

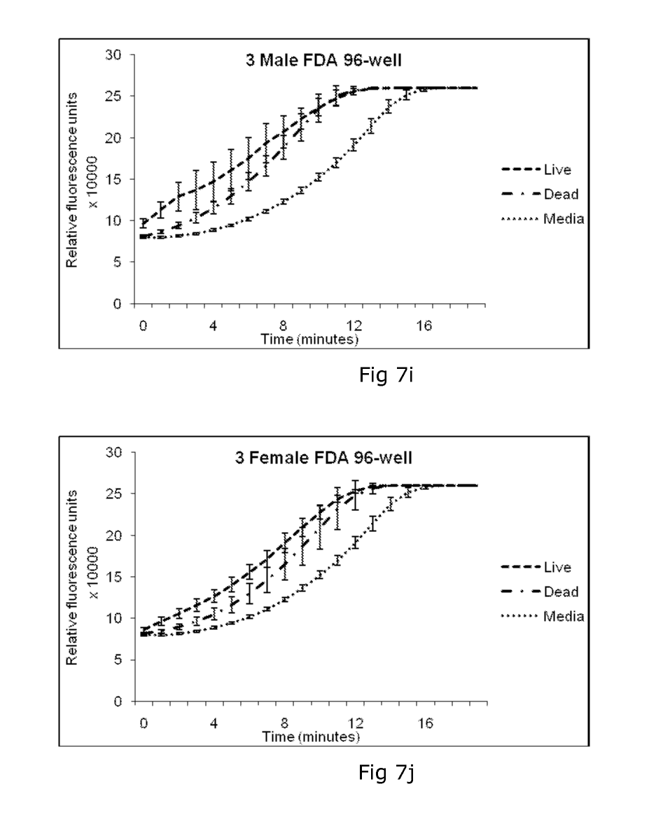

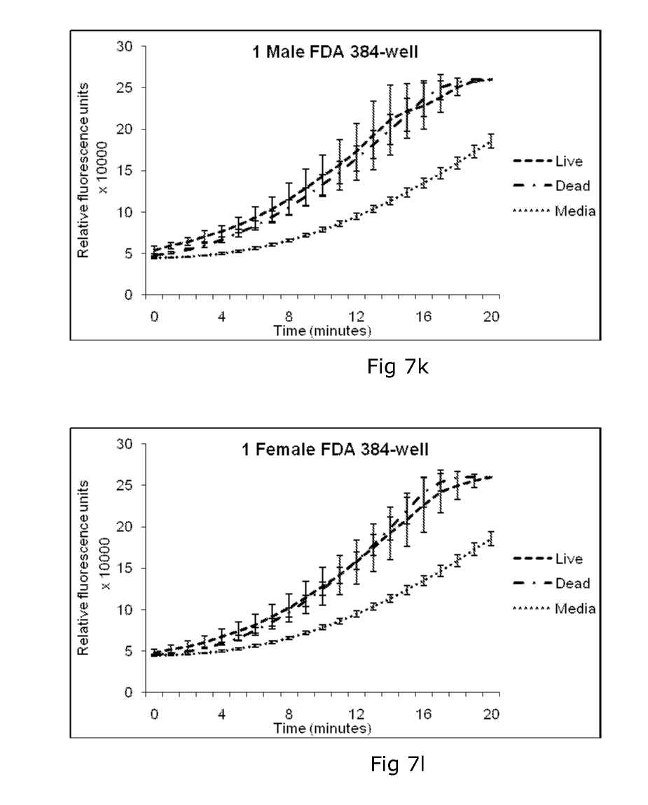

To further the applicability of this assay for use with the parasite Schistosoma mansoni, a second lifestage was tested. The 7-week adult parasites were harvested by perfusion from mice and were cultured for 24 hours before assaying. Dead worms were created by the addition of 10 μM auranofin to the culture medium at the start of the 24 hours. Worms were cultured at 1 and 3 worms per well in a 96 well plate or at 1 worm per well in a 384 well plate. Males and females were cultured separately to determine if there would be any fluorescent differences between them. The results are shown in FIGS. 7a to 7l.

For both PI and FDA there were strong differences between live and dead worms for all experiments. However, there was an increased fluorescence for live females, which is hypothesised to be auto-fluorescence of the vitellaria.

Removal of Wash Step

A limiting step in the speed of the assay is the need to include a wash step before the addition of the dyes in order to limit possible background fluorescence from FDA.

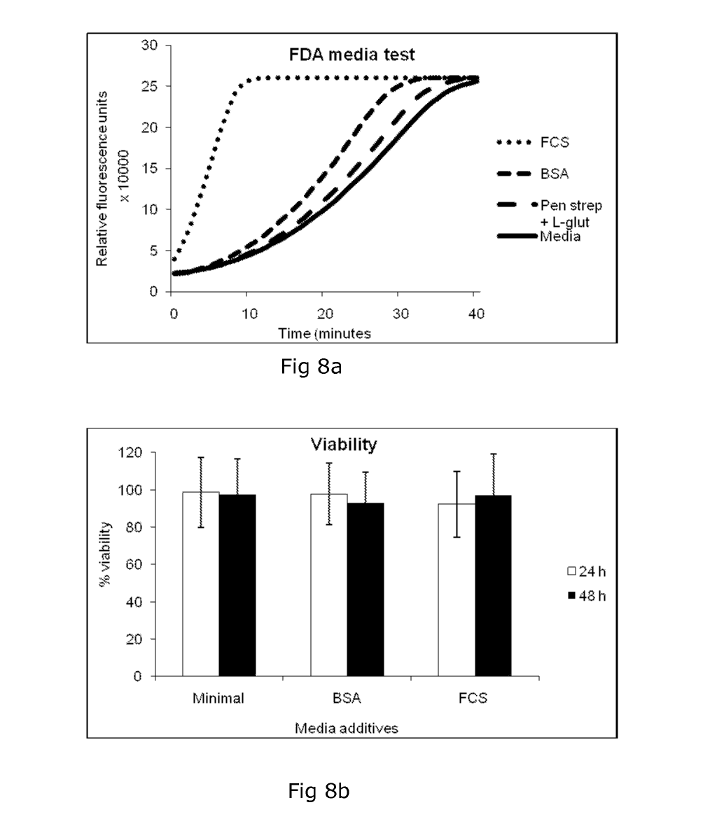

A test of media additives over time revealed that FCS is the main contributor of this fluorescence. It was unknown at the time of this test if schistosomula could survive for 48 hours without FCS. BSA was tested as a possible medium alternative and found to exhibit more favourable fluorescent characteristics compared to FCS. The results are shown in FIG. 8a.

Following on from the test of media additives, schistosomula were cultured for 24 or 48 hours in media containing either FCS or BSA and compared to parasites cultured in media containing neither of these additives.

At neither time point were there significant differences in viability between any of the treatments. This result allowed us to consider not adding either FCS or BSA to the culture medium. The results are shown in FIG. 8b.

Reduction of Time from 48 Hours to 24 Hours

All the original data for the assay was collected on schistosomula that had been cultured for 24 hours prior to the addition of test compounds, followed by a further 24 hour incubation. To investigate the reduction of the assay from 48 hours to 24 hours, the use of 3 hour versus 24 hour schistosomula was tested.

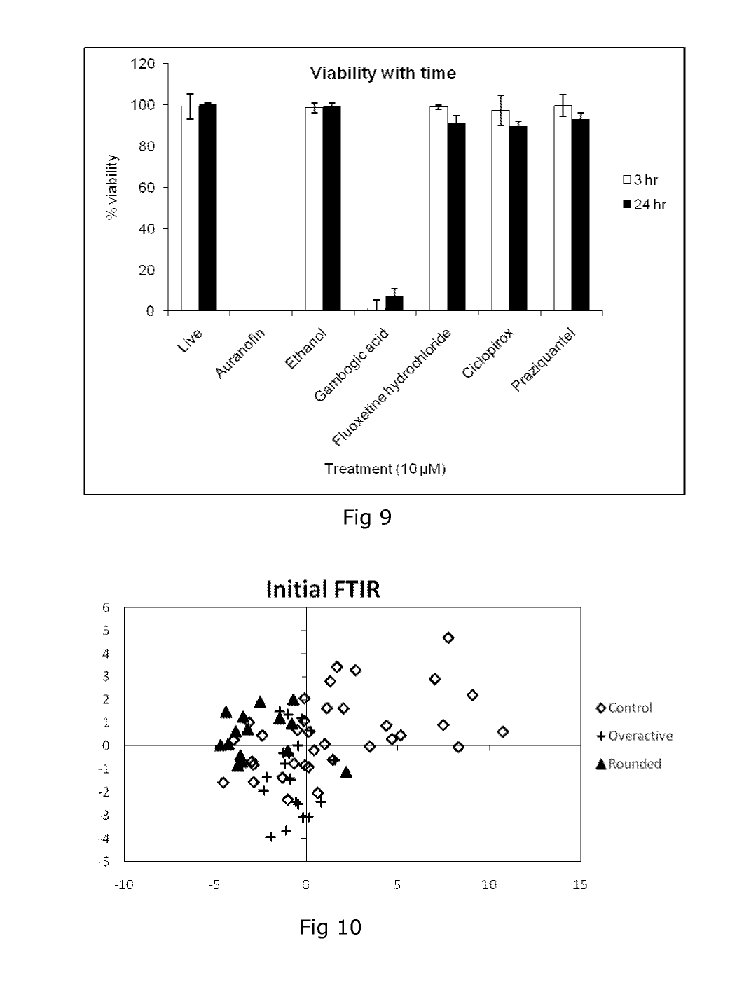

This test was run in media that did not contain FCS. The results are shown in FIG. 9.

There was no significant change in viability (in response to a selection of test reagents) between 3 hour and 24 hour schistosomula, so the initial 24 hour incubation was considered unnecessary.

One concern with the removal of FCS from the culture media was that there would be a decrease in viability in schistosomula that were stressed by exposure to previously non-lethal chemicals. While this test does not represent a detailed investigation of this point, it does provide some evidence that the lack of FCS does not cause a decrease in the viability of schistosomula treated with a variety of compounds.

A Non-Lethal Endpoint Readout—e.g. Fourier Transform Infrared Spectrometry (FTIR) Investigation

While the dual-fluorescent viability assay provides a fast, objective and reliable means of determining the viability of schistosomula, it does not allow identification of compounds that cause a phenotype other than death. An example of this is praziquantel, which does not kill in vitro but does cause schistosomula to display a strong rounded phenotype (FIG. 6). Many other examples exist of anti-nematode, as well as anti-platyhelminth, compounds that do not cause death but rather cause abnormal phenotypes such as rounded shapes, hyperactivity or paralysis and are desirable as antiparasitic drugs for these reasons.

In an effort to remedy this omission, Fourier Transform Infrared Spectrometry (FTIR) was investigated as a means of rapidly gaining information on the metabolic footprint of altered phenotypes. FTIR is proposed as an additional readout to fluorescence for this assay and as an example of a readout for ‘non-lethal outcomes’.

An initial experiment was set up using 4 compounds with an overactive phenotype, 3 compounds with a rounded phenotype and 3 compounds that displayed no obvious phenotype. As shown in FIG. 10, the data from this experiment is promising, with the rounded and the overactive schistosomula tending to group.

Haemonchus Contortus FTIR

Parasite Culture and Treatment

1. Haemonchus contortus L3 larvae were exsheated in 5% sodium hypochlorite for 10 minutes.

2. Following exsheathment the larvae were pelleted by centrifugation at 200×g for 2 minutes, the supernatant was removed and replaced with 1×PBS. This wash step was repeated twice to remove any traces of sodium hypochlorite then resuspended in DMEM containing L-glutamine and pen-strep but lacking in phenol red.

3. The larvae were aliquoted into a 384 well plate at approx. 100 larvae per well.

4. Compounds to be tested (aldicarb, levamisole and ivermectin) were made up at 3 mg/ml in DMSO then added to test wells at 1/100.

5. An identical quantity of DMSO, lacking any compound, was added to control wells.

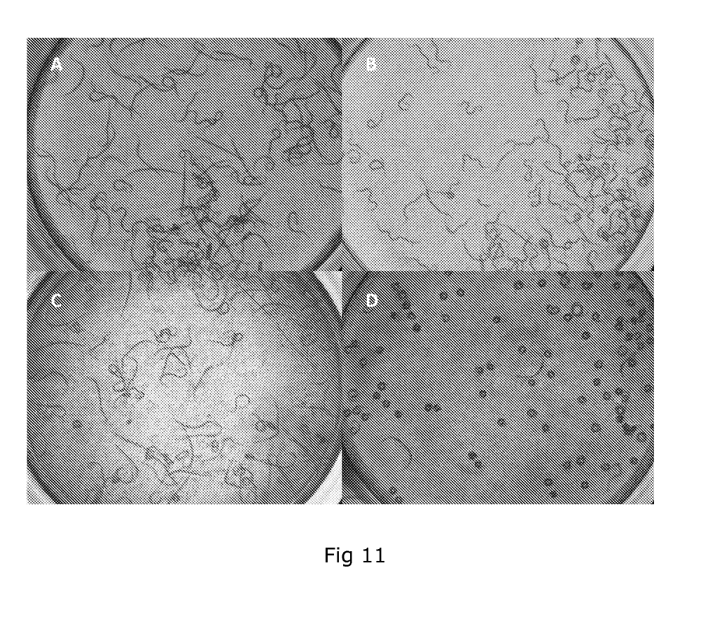

6. The larvae were cultured for 24 hours at 37° C. following the addition of compounds (see FIG. 11). Wells containing media and compounds, but lacking in larvae, were also set up as controls for background.

FTIR Assay Setup

For FTIR, two samples were run from each well containing larvae. The first consisted of 10 μl of supernatant taken from the top of the well without any disturbance. The second consisted of 10 μl containing worms and any sediment stirred up from the bottom of the well. A third 10 μl sample of media containing compounds but no larvae was also run for elimination of drug chemistry. Samples were dried at 50° C. for 45 minutes to prevent interference from water before being loaded into the FTIR machine.

Data Analysis

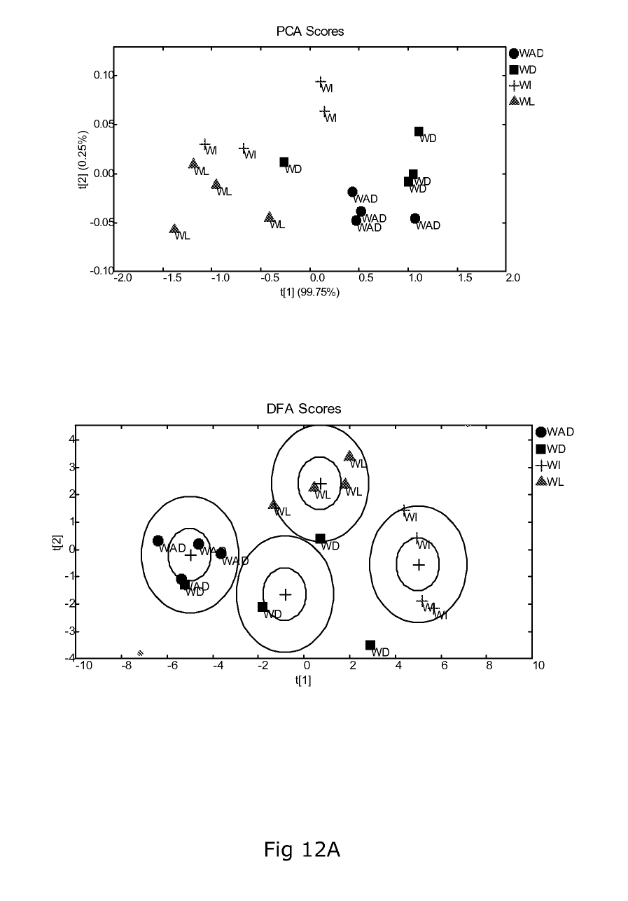

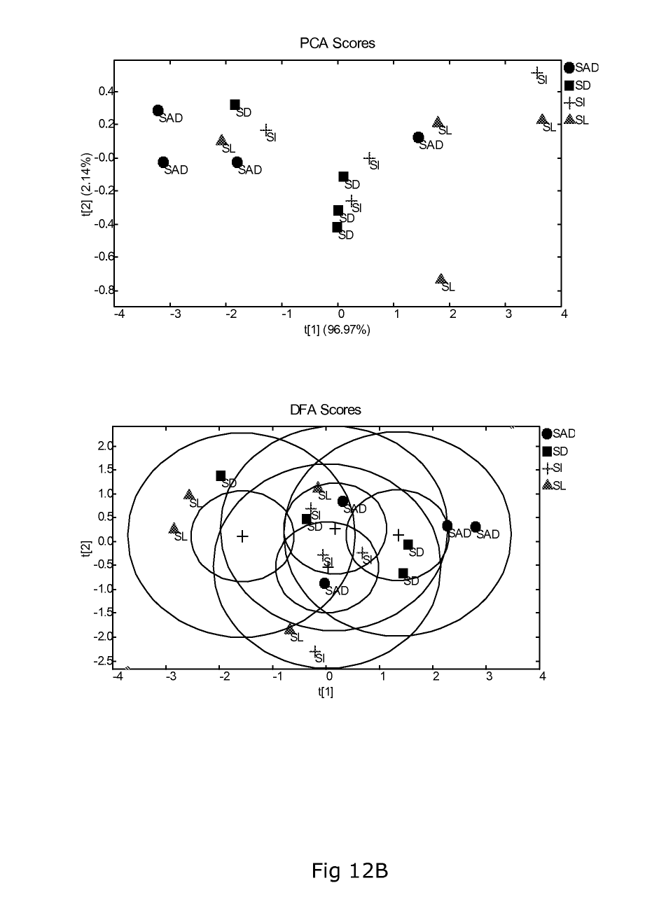

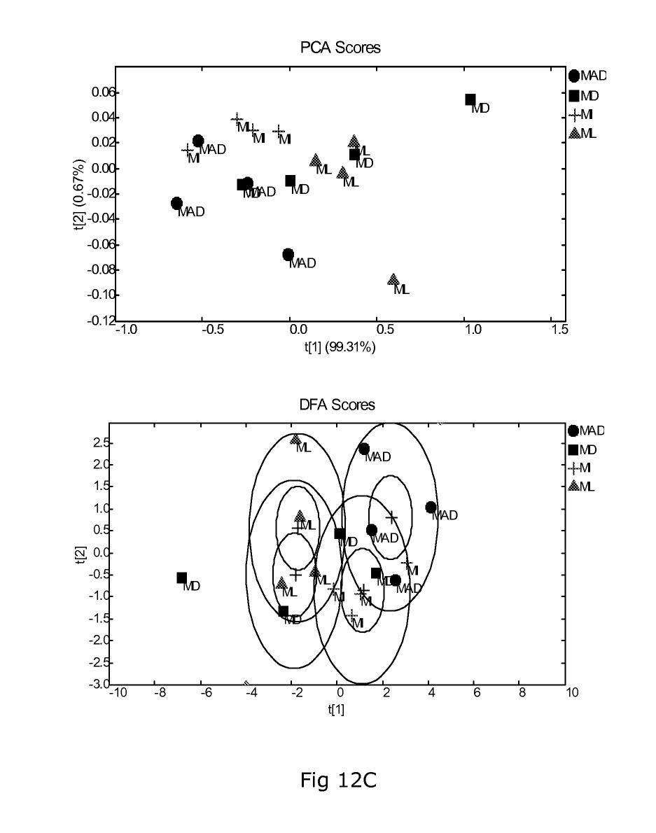

Collected spectra were analysed using PyChem software. Two analyses were run for all data: principle components analysis (PCA; which compresses all of the data into smaller, discreet (2) components without there being any assumptions made) and discriminate function analysis (DFA; which takes information provided about class structure to run an algorithm to maximise distances between groups (different drug treatments) and minimise distances within groups (replicates of the same drug treatment)). The circles on the DFA graphs represent mean group centres i.e. treatments that segregate with confidence intervals of 95% (inner circle) and 99% (outer circle). See FIG. 12.

Conclusions/Observations

FTIR data clustering is seen between the treatments for worms when FTIR is used. These data clusters correspond to the three discreet, drug-induced worm phenotypes observed by microscopy.

No clear discrimination of control FTIR spectra collected from media (containing just the drugs) indicates that the differences seen in worm data are not caused by compound chemistry.

With regard to the results presented in FIGS. 12A to 12C, we have found these patterns to be repeatable (in three independent experiments), although the number of worms being cultured seems to be important. Too few worms and the effects are no longer as clear.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications are covered by the appended claims. The content of all references mentioned herein are incorporated herein by reference in their entirety.

1. A method for evaluating the viability of parasitic or non-parasitic worms, for example helminths, comprising
(a) providing a sample comprising one or more parasitic or non-parasitic worms, for example helminths;
(b) contacting the parasitic or non-parasitic worms with a concentration of a first fluorescent dye sufficient to yield a detectable first colour fluorescence in any live parasitic or non-parasitic worms present in the sample and a concentration of a second fluorescent dye sufficient to yield a detectable second colour fluorescence in any dead parasitic or non-parasitic worms present in the sample; and
(c) detecting the first and second fluorescence of the parasitic or non-parasitic worms.

2. A method for screening for the effect of one or more agents or an event on the viability of parasitic or non-parasitic worms, for example helminths, comprising the method according to claim 1, further comprising contacting the parasitic or non-parasitic worms with one or more test agents, or subjecting the parasitic or non-parasitic worms to the event, prior to the step of contacting the parasitic or non-parasitic worms with the fluorescent dyes.

3. A method according to claim 1, wherein the first fluorescent dye is fluorescein diacetate that yields a detectable green fluorescence in any live parasitic or non-parasitic worms present in the sample.

4. A method according to claim 1, wherein the second fluorescent dye is propidium iodide that yields a detectable red fluorescence in any dead parasitic or non-parasitic worms present in the sample.

5. A method according to claim 1, further comprising allowing sufficient time for the two dyes to yield optimal fluorescence.

6. A method according to claim 1, comprising detecting the first fluorescence and the second fluorescence sequentially.

7. A method according to claim 6, comprising detecting the first colour fluorescence before the second colour fluorescence.

8. A method according to claim 1, wherein the first fluorescent dye is fluorescein diacetate and the method comprises detecting the green fluorescence about 4 to about 8 minutes after contact with the sample comprising parasitic or non-parasitic worms.

9. A method according to claim 1, wherein the second fluorescent dye is propidium iodide and the method comprises detecting the red fluorescence about 10 to about 35 minutes after contact with the sample comprising parasitic or non-parasitic worms.

10. A method according to claim 9, comprising detecting the red fluorescence about 14 to about 29 minutes after contact with the sample comprising parasitic or non-parasitic worms.

11. A method according to claim 2, further comprising removing the one or more test agents or the event prior to contacting the parasitic or non-parasitic worms with the fluorescent dyes.

12. A method according to claim 1, further comprising providing the parasitic or non-parasitic worms in a culture media, preferably, wherein the culture media does not contain FCS.

13. A method according to claim 12, comprising removing at least a proportion of the culture media prior to contacting the parasitic or non-parasitic worms with the fluorescent dyes.

14. A method according to claim 12, further comprising adding an esterase inhibitor to the sample comprising the parasitic or non-parasitic worms and culture media prior to contacting the parasitic or non-parasitic worms with the fluorescent dyes in a concentration sufficient to prevent hydrolysis of the first fluorescent dye outside of the parasitic or non-parasitic worm cell membrane.

15. A method according to claim 1, further comprising providing a microtiter plate comprising a plurality of wells, each of the wells containing one or more parasitic or non-parasitic worms.

16. A method according to claim 15, wherein the microtiter plate is selected from the group consisting of a 12-well plate, a 24-well plate, a 96-well plate, a 384-well plate, and a 1536-well plate, preferably, the plate has 96 wells or 384 wells.

17. A method according to claim 15 or claim 16, wherein the wells each contain between 1 and about 15 adult parasitic or non-parasitic worms, preferably between about 5 and about 10.

18. A method according to claim 15 or claim 16, wherein
(i) the microtiter plate has 96 wells each containing between about 100 and about 1000 immature parasitic or non-parasitic worms, preferably between about 500 and about 1000, or
(ii) the microtiter plate has 384 wells each containing between about 50 and about 500 immature parasitic or non-parasitic worms, preferably between about 100 and about 300 immature parasitic or non-parasitic worms.

19. A method according to claim 18, wherein the immature parasitic or non-parasitic worms are schistosomula.

20. A method according to claim 15, wherein the fluorescence is detected by means of a multi-well fluorescent plate reader.

21. A method according to claim 1, wherein the fluorescence is detected by means selected from: flow cytometry, fluorescent microscopy and fluorescence spectroscopy.

22. A method according to claim 1, wherein the parasitic or non-parasitic worms are helminths, preferably platyhelminthes.

23. A method according to claim 1, wherein the parasitic or non-parasitic worms are nematodes.

24. A method according to claim 1, wherein the parasitic or non-parasitic worms are immature adults.

25. A method according to claim 24, wherein the immature adults are schistosomula or Haemonchus contortus L3 larvae.

26. A method according to claim 1 which comprises contacting the sample comprising one or more parasitic or non-parasitic worms with one or more test agents or subjecting the sample to an event, wherein the method is for detecting phenotypic and/or metabolic changes in a sample of living parasitic or non-parasitic worms and further comprises:
(I) performing FTIR spectral analysis of the sample; and
(II) comparing the spectra produced from the sample with an FTIR spectra from one or more reference standards indicative of abnormal phenotype and/or metabolic change to identify the presence of one or more abnormal phenotypes and/or metabolic changes in the sample.

27. A method for detecting phenotypic and/or metabolic changes in a sample of living parasitic or non-parasitic worms, for example helminths following contact with one or more test agents or after subjecting the sample to an event, the method comprising
(a) providing a sample comprising one or more parasitic or non-parasitic worms, for example helminths;
(b) contacting the parasitic or non-parasitic worms with one or more test agents or subjecting the sample to an event;
(c) performing FTIR spectral analysis of the sample; and
(d) comparing the spectra produced from the sample with an FTIR spectra from one or more reference standards indicative of abnormal phenotype and/or metabolic change to identify the presence of one or more abnormal phenotypes and/or metabolic changes in the sample.

28. A method according to claim 26, wherein FTIR spectral analysis is performed on the supernatant of the sample comprising one or more parasitic or non-parasitic worms, and/or on worm bodies of the sample comprising one or more parasitic or non-parasitic worms.

29. A kit for evaluating the viability of parasitic or non-parasitic worms, for example helminths, the kit comprising:
(i) a microtiter plate comprising a plurality of wells;
(ii) a culture media;
(iii) a first fluorescent dye that yields a detectable first colour fluorescence in any live parasitic or non-parasitic worms; and
(iv) a second fluorescent dye that yields a detectable second colour fluorescence in any dead parasitic or non-parasitic worms.

30. A kit according to claim 29, further comprising a wash medium.

31. A kit according to claim 29, further comprising a positive control anti-helminth agent selected from: auronofin, ethanol, phenol, iodine, methanol, formaldehyde, dimethyl sulphate, hydrogen peroxide, hydrochloric acid, acetic acid, formic acid and sodium dodedecyl sulphate.

32. A kit according to claim 29, wherein the first fluorescent dye is fluorescein diacetate.

33. A kit according to claim 29, wherein the second fluorescent dye is propidium iodide.

34. A kit according to claim 29, wherein the culture media is lyophilised.

35. A kit according to claim 30, wherein the wash medium is in lyophilised or liquid form.

36. A kit according to claim 29, further comprising a plurality of parasitic or non-parasitic worms, for example helminths.

37. A kit according to claim 36, wherein the plurality of parasitic or non-parasitic worms are disposed such that each of a plurality of the wells contains one or more of the parasitic or non-parasitic worms.

38. A kit according to claim 29, further comprising instructions for use of the kit for high-throughput screening for the effect of one or more test agents or an event on the viability of a parasitic or non-parasitic worm, for example a platyhelminth or nematode.

39. A kit for evaluating phenotypic and/or metabolic changes of parasitic or non-parasitic worms, for example helminths following contact with one or more test agents or after subjecting a sample of parasitic or non-parasitic worms, for example helminths to an event, the kit comprising:
(i) a microtiter plate comprising a plurality of wells;
(ii) a culture media; and
(iii) instructions for performing FTIR analysis.
